(19) United States
(12) Patent Application Publication
(10) Pub. No.: US 2015/0320321 A1
(43) Pub. Date: Nov. 12, 2015

(54) OBJECT INFORMATION ACQUIRING APPARATUS

(71) Applicant: CANON KABUSHIKI KAISHA, Tokyo (JP)

(72) Inventor: Kenji Oyama, Tokyo (JP)

(21) Appl. No.: 14/700,310

(22) Filed: Apr. 30, 2015

(30) Foreign Application Priority Data
May 7, 2014 (JP) 2014-096162

(51) Int. Cl. A61B 5/00

(52) U.S. Cl. CPC A61B 5/0095; A61B 5/708; A61B 5/4312

(57) ABSTRACT

An object is inserted through a first opening of a bed, and a probe is disposed which includes a breast support member supporting a plurality of first acoustic wave detection elements, which receive an acoustic wave propagated from the object and output a first reception signal so that the first acoustic wave detection elements are oriented to a predetermined area. The probe is separated from the object inserted through the first opening of the bed in the normal direction of the opening surface of the first opening. The bed holds a plurality of second acoustic wave detection elements that receive the acoustic wave propagated from the object and output the second reception signal, and orient the second acoustic wave detection elements to the predetermined area.

### BACKGROUND OF THE INVENTION

1. Field of the Invention

The present invention relates to an object information acquiring apparatus that visualizes object information using information on an acoustic wave that propagates from the object.

2. Description of the Related Art

Techniques to acquire biological functional information using light and ultrasonic waves (hereafter called “photoacoustic tomography” or “PAT”) have been proposed.

If pulsed light of a visible light, a near infrared light or the like is irradiated onto biological tissue, light absorbing substance in the biological tissue, especially such substance as hemoglobin in blood, absorbs energy of the pulsed light, instantaneously expands, and generates a photoacoustic wave (typically ultrasonic wave) as a result. This phenomena is called “photoacoustic effect”, and PAT is used for visualizing information of the biological tissue by measuring a photoacoustic wave. By visualizing a light energy absorption density distribution (density distribution of light absorbing substance in the biological tissue, which is a source of generating a photoacoustic wave), an image of active angiogenesis due to cancer tissue can be formed. Further, functional information, such as the oxygen saturation of blood, can be acquired using the dependency of the generated photoacoustic wave on the light wavelength.

Moreover, in PAT techniques, light and an ultrasonic wave are used for imaging biological information, therefore non-invasive image diagnosis, without exposure to radiation, can be performed, which is a great advantage in terms of lessening burden on patients. Therefore the active use of PAT in screening breast cancer and early diagnosis is expected, instead of using an X-ray apparatus where repeated diagnosis is difficult.

For example, an object information acquiring apparatus that acquires object information in a wide range, by implementing mechanical scanning with a probe for measuring a photoacoustic wave propagating from the object, has been proposed. According to this apparatus, the photoacoustic wave is measured by a mechanical scan type probe and a fixed type probe, whereby the visualization area of the object information can be further expanded (International Publication No. WO 2013/046437).

Another proposed technique is acquiring object information in a wide range by implementing mechanical scanning with a probe constituted by a plurality of transducers which are disposed at different positions along an approximately spherical crown shape. According to this technique, a solid angle to measure the photoacoustic wave with respect to the object can be increased by using the approximately spherical crown shape, whereby the object information can be visualized at high accuracy. Moreover, by turning the reception directivity of the plurality of transducers, disposed along the approximately spherical crown shape, toward a predetermined area, the predetermined area can be visualized at high accuracy (U.S. Pat. No. 6,104,942).

Patent Literature 1: International Publication No. WO 2013/046437

Patent Literature 2: U.S. Pat. No. 6,104,942

### SUMMARY OF THE INVENTION

To accurately visualize the distribution of light absorbing substance, which exists in biological tissue based on the time when a photosensitive wave that propagates from the object is received and the intensity of the acoustic wave, it is preferable to secure a solid angle that is as large as possible by disposing a plurality of probes or transducers thereof at different positions. If the measurement points are polarized or if the solid angle is insufficient in the arrangement of the transducers, a virtual image (artifact) that does not actually exist stands out in the specific direction corresponding to the insufficient measurement directions when object information is imaged.

According to the technique disclosed in International Publication No. WO 2013/046437, if the movable probe that mechanically scans and the fixed probe disposed between the movable probe and the object measure the photoacoustic wave in the same object area, insufficiency of the solid angles can complement each other in the respective measurement.

However a probe has finite directivity in the acoustic wave detection capability thereof, hence the area, where the measurement area measured by the fixed probe is superposed on the measurement positions during the scanning by the movable probe, is limited.

According to the technique disclosed in U.S. Pat. No. 6,104,942, a probe constituted by a plurality of transducers disposed at different positions along the approximately spherical crown shape is used, therefore a solid angle, close to half of the entire solid angle, can be secured and measured. Further, in the case of a probe having an approximately spherical crown shape, a predetermined area that can be visualized at high accuracy can be formed in accordance with the arrangement of the transducers and the directivity of the transducers. For example, if orientations of all the transducers are directed to the center of the curvature of the approximately spherical crown shape, the predetermined area can be formed at the center of the curvature. By mechanically scanning this area, the object information in a wide range can be visualized at high sensitivity and high resolution. The orientation of the transducer here refers to the direction in which the transducer is oriented, that is, the direction in which the reception sensitivity of the transducer is highest.

To visualize the object information at high accuracy, it is preferable to measure the photoacoustic wave at a solid angle that is as large as possible, as mentioned above.

However, in the technique disclosed in U.S. Pat. No. 6,104,942, the probe is disposed at a position that does not physically interfere with the object in the entire scanning range, in order to allow mechanical scanning of the probe, which has an approximately spherical crown shape, on the object. Based on this positional relationship, a part or all of the object is outside the opening surface of the probe which has an approximately spherical crown shape, which means that the area that can be visualized at high precision must be formed outside the opening space. As a result, the solid angle of the approximately spherical crown shape, that is, the solid angle of the measurement points, is restricted, which tends to make an artifact stand out during imaging.

With the foregoing in view, it is an object of the present invention to suppress an artifact that is generated when the object information is visualized by measuring the photoacoustic wave using the probe constituted by a plurality of transducers disposed at different positions, so as to orient to the predetermined area.

To solve this problem, the present invention provides an object information acquiring apparatus, comprising:

a bed configured to have a first opening through which an object is inserted;

a probe configured to include a plurality of first acoustic wave detection elements receiving an acoustic wave propagated from the object and outputting a first reception signal, and a breast support member supporting the plurality of first acoustic wave detection elements so that a direction in which reception sensitivity of at least a part of the plurality of first acoustic wave detection elements is the highest, and a direction in which reception sensitivity of the first acoustic wave detection elements different from the part of the first acoustic wave detection elements is the highest, are different and are both oriented to a predetermined area, the probe being separated from the object, which is inserted through the first opening of the bed, in the normal direction of an opening surface of the first opening;

a plurality of second acoustic wave detection elements which are disposed between the bed and the probe, and of which highest sensitive direction is oriented to the predetermined area, and moreover which receive the acoustic wave and output the second reception signal;

a first position controller configured to change a positional relationship between the object and the predetermined area by changing a positional relationship between the object and the probe; and

a generator configured to generate object information based on the first and second reception signals.

The present invention also provides an object information acquiring apparatus, comprising:

a bed configured to have a first opening through which an object is inserted;

a probe configured to include a plurality of first acoustic wave detection elements receiving an acoustic wave propagated from the object and outputting a first reception signal, and a breast support member supporting the plurality of first acoustic wave detection elements so that orientation axes of the plurality of first acoustic wave detection elements are collected, the probe being separated from the object, which is inserted through the first opening of the bed, in the normal direction of an opening surface of the first opening;

a plurality of second acoustic wave detection elements which are disposed between the bed and the probe, and of which highest sensitive direction is oriented to a predetermined area, and moreover which receive the acoustic wave and output the second reception signal;

a first position controller configured to change a positional relationship between the object and the predetermined area by changing a positional relationship between the object and the probe; and

a generator configured to generate object information based on the first and second reception signals

The present invention also provides an breast information acquiring apparatus, comprising:

a bed having an opening through which an breast is inserted, wherein the bed is configured to support a examinee;

a breast support member configured to be located below the opening and connected to the bed so as to form an upper space in which the breast can be inserted and a lower space in which an acquiring unit is located,

wherein the acquiring unit comprises;

a probe unit located below the breast support member and be movably arranged respect to the opening, wherein the probe unit having a moving acoustic wave detection element which detects an acoustic wave propagated from the breast, and having a moving bowl-shaped support member on which the moving acoustic wave detection element is fixed in concave portion such that the moving acoustic wave detection element and the moving bowl-shaped support member are integrally moved respect to the opening;

a fixed acoustic wave detection element fixed on the bed and located at a position between the bed and the probe unit so that a highest sensitive direction of the fixed acoustic wave detection element and a highest sensitive direction of the moving acoustic wave detection element overlap each other;

a position controller configured to change a relative position between the breast support member and the probe unit; and

a generator configured to generate object information based on a detecting signal of the moving acoustic wave detection element and a detecting signal of the fixed acoustic wave detection element.

As described above, according to the present invention, an artifact that is generated when the object information is visualized can be suppressed by measuring the photoacoustic wave using the probe constituted by a plurality of transducers disposed at different positions, so as to orient to the predetermined area.

Further features of the present invention will become apparent from the following description of exemplary embodiments with reference to the attached drawings.

### BRIEF DESCRIPTION OF THE DRAWINGS

FIG. 1 is a schematic diagram depicting Example 1 of an object information acquiring apparatus of the present invention;

FIG. 2A and FIG. 2B are conceptual diagrams depicting a configuration of a probe according to Example 1;

FIG. 3A and FIG. 3B are conceptual diagrams depicting the reception characteristic of the probe according to Example 1;

FIG. 4A is a diagram depicting an overview of image reconstruction according to Example 1;

FIG. 4B is a conceptual diagram depicting a tendency of an artifact that is generated by a semispherical probe;

FIG. 4C is a conceptual diagram depicting the tendency of an artifact that is generated by a probe according to Example 1;

FIG. 5A and FIG. 5B are conceptual diagrams depicting a configuration of fixed transducers according to Example 1;

FIG. 6 is a flow chart depicting a flow of object information acquisition according to Example 1;

FIG. 7 is a conceptual diagram depicting two-dimensional scanning control of the probe according to Example 1;

FIG. 8A and FIG. 8B are conceptual diagrams depicting an artifact suppression effect according to Example 1;

FIG. 9 is a schematic diagram depicting Example 2 of the object information acquiring apparatus of the present invention;

FIG. 10 is a flow chart depicting a flow of object information acquisition according to Example 2;

FIG. 11A to FIG. 11C are conceptual diagrams depicting position control of fixed transducers according to Example 2;

FIG. 12 is a schematic diagram depicting Example 3 of the object information acquiring apparatus of the present invention;

FIG. 13 is a flow chart depicting a flow of object information acquisition according to Example 3;

FIG. 14A to FIG. 14C are conceptual diagrams depicting attitude control of the rotation angles of fixed transducers according to Example 3; and

FIG. 15A to FIG. 15C are conceptual diagrams depicting attitude control of the elevation/depression angles of the fixed transducers according to Example 3.

### DESCRIPTION OF THE EMBODIMENTS

Embodiments of the present invention will now be described with reference to the drawings. As a rule, a same composing element is denoted with a same reference numeral, where redundant description is omitted. A detailed calculation formula, calculation procedure or the like that are described herein below should be changed in accordance with the configuration of the apparatus and various conditions, and are not intended to limit the scope of the invention to the description herein below.

The object information acquiring apparatus of the present invention includes an apparatus using ultrasonic echo technology that transmits an ultrasonic wave to an object, receives a reflected wave (echo wave) reflected inside the object, and acquires the object information as image data. The object information acquiring apparatus of the present invention also includes an apparatus that uses a photoacoustic effect, that receives an acoustic wave which is generated in an object by irradiating light (electromagnetic wave), such as near infrared, to the object, and acquires the object information as image data.

In the case of the former apparatus that uses ultrasonic echo technology, the object information to be acquired is information reflecting the difference of acoustic impedance of the tissue inside the object. In the case of the latter apparatus that uses the photoacoustic effect, the object information to be acquired is the generation source distribution of the acoustic wave which is generated by light irradiation, the initial sound pressure distribution inside the object or the absorption density distribution/absorption coefficient distribution of the light energy derived from the initial sound pressure distribution, and the concentration distribution of the substance constituting the tissue. The concentration distribution of the substance is, for example, oxygen saturation distribution, total hemoglobin concentration distribution, or oxy-/deoxy-hemoglobin concentration distribution.

The characteristic information, which is object information at a plurality of positions, may be acquired as two-dimensional or three-dimensional characteristic distribution. The characteristic distribution can be generated as image data that indicates the characteristic information inside the object.

The acoustic wave in the present invention typically is an ultrasonic wave, including an elastic wave called a “sound wave” and an “ultrasonic wave”. An acoustic wave generated by the photoacoustic effect is called a “photoacoustic wave” or an “optical ultrasonic wave”. An acoustic detector (e.g. probe) receives an acoustic wave that is generated or reflected inside the object.

### Example 1

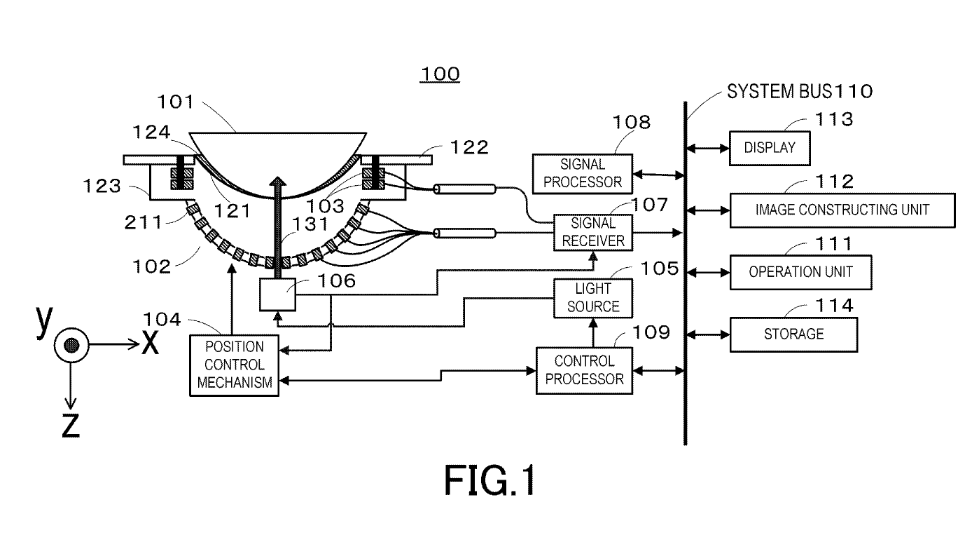

FIG. 1 is a schematic diagram depicting Example 1 of an object information acquiring apparatus 100 (hereafter called “apparatus 100”) according to an embodiment of the present invention. The apparatus 100 in Example 1 has the following: a movable probe 102 that receives a photoacoustic wave propagated from an object 101 and outputs a photoacoustic wave signal which is a first reception signal; and fixed transducers 103 which are second acoustic wave detection elements that output a second reception signal. The movable probe 102 includes a plurality of transducers 211, which are first acoustic wave detection elements that output a photoacoustic wave signal which is the first reception signal. Transducers 211 are called “moving acoustic wave detection elements”, too.

The photoacoustic signal is an analog signal. The apparatus 100 also includes a position control mechanism 104 which is a position controller that controls a position of the movable probe 102, a light source 105 that generates light, and an irradiation optical system 106 that irradiates light onto the object 101. Furthermore, the apparatus 100 includes a signal receiver 107 that amplifies the photoacoustic wave signal from the movable probe 102 and the fixed transducers 103, performs A/D conversion, and transmits a photoacoustic digital signal, and a signal processor 108 that performs accumulating for the photoacoustic wave digital signal. Here the generator includes the signal receiver 107 and the signal processor 108, and generates object information based on the accumulation result of the photoacoustic wave digital signal.

The apparatus 100 further includes: an operation unit 111 that the user (primarily medical technologist) uses to input the instructions to start imaging or the like and the parameters required for imaging to the apparatus 100; and an image constructing unit 112 that creates an image of the object information acquired by the generator. The apparatus 100 also includes a display 113 that displays the generated images and a user interface (UI) to operate the apparatus.

The apparatus 100 further includes a control processor 109 that accepts various operations by the user via the operation unit 111, generates control information required for generating the target object information, and controls each function via a system bus 110. The apparatus 100 also includes a storage 114 that stores the acquired photoacoustic wave digital signal, the generated image, and other information on operation. The object 101 to be imaged is a breast in the case of breast cancer diagnosis in a Breast Oncology Department.

Each composing element of the apparatus 100 will now be described in detail.

Position Control Mechanism 104

The position control mechanism 104 is constituted by a drive member, such as a motor, and mechanical parts to transfer the drive force thereof, and controls the pulsed light 131 and the movable probe 102 according to the scanning control information from the control processor 109. By repeating the signal acquisition while two-dimensionally scanning the positions of the pulsed light 131 and the movable probe 102 with respect to the object 101, target object information in a wide range can be acquired. The position control mechanism 104 outputs the current position control information to the control processor 109, synchronizing with the emission control by the irradiation optical system 106 to emit pulsed light 131 each time.

Light Source 105

The light source 105 emits a pulsed light (width: 100 nm or less) having a central wavelength in the near infrared region. For the light source 105, a solid-state laser that can emit pulses having a central wavelength in the near infrared region (e.g. Yttrium-Aluminum-Garnet laser, Titan-Sapphire laser) is normally used. Such a laser as a gas laser, dye laser and semiconductor laser can also be used, and a light emitting diode or the like may be used for the light source 105 instead of a laser.

The wavelength of the light is selected according to the light absorbing substance in the biological tissue to be measured, such as oxy-hemoglobin, deoxy-hemoglobin, in a malignant tumor that contains many blood vessels or newly generated blood vessels containing a volume of such hemoglobins, glucose, cholesterol and the like. For example, if the measurement target is hemoglobin in newly generated blood vessels of a breast cancer, light in a 600 to 1000 nm wavelength is absorbed, and the light absorption of water constituting the biological tissue reaches the minimum at around an 830 nm wavelength, therefore the light absorption in a 750 nm to 850 nm range is relatively large. The light absorptivity changes depending on the wavelength of the light due to the state of hemoglobin, that is, due to oxygen saturation, therefore the functional changes of the biological tissue can also be measured using this wavelength dependency.

Irradiation Optical System 106

The irradiation optical system 106 guides the pulsed light emitted from the light source 105 to the object, generates a light 131 suitable for acquiring the signal, and emits the generated light. The irradiation optical system 106 typically is constituted by optical components, such as a lens or prism, to collect or expand light, a mirror to reflect light, and a diffusion plate to diffuse light. Such an optical wave guide as an optical fiber may also be used to guide light from the light source 105 to the irradiation optical system 106. As the standard to irradiate a laser light onto the skin and into the eyes, IEC 60825-1 specifies the maximum permissable exposure, and includes such conditions as the wavelength of light, the exposure duration and the pulse repetition. The irradiation optical system 106 generates the light 131 that satisfies this standard for the object 101.

The irradiation optical system 106 has an optical configuration (not illustrated) that detects the emission of the light 131 onto the object 101, and generates a synchronization signal for receiving the photoacoustic wave signal and recording in sync with the detection. The emission of the light 131 can be detected, for example, by splitting a part of the pulsed light generated by the light source 105 by using such an optical system as a half mirror, guiding the light to the optical sensor, and the optical sensor generating a detection signal based on the guided light. If a fiber bundle is used to guide the pulsed light, a part of the fiber can be branched and guided to the optical sensor for detection. The synchronization signal generated by this detection is inputted to the signal receiver 107 and the position control mechanism 104.

Signal Receiver 107

The signal receiver 107 amplifies the photoacoustic wave signals generated by the movable probe 102 and the fixed transducers 103 according to the synchronization signal inputted from the irradiation optical system 106, and converts the amplified photoacoustic wave signal into photoacoustic wave digital signals. The signal receiver 107 is constituted by: a signal amplifying unit (not illustrated) that amplifies analog signals generated by the movable probe 102 and the fixed transducers 103; and an A/D conversion unit (not illustrated) that converts analog signals into digital signals. The signal receiver 107 described in Example 1 is assumed to have a configuration that allows receiving signals of all the transducers of the transducers 211 and the fixed transducer 103. The present invention is not limited to this configuration, but may have independent hardware for each transducer. The movable probe 102 is arranged to be located below the support member 121 when the movable probe 102 moves, thus the movable probe 102 have a portion which is located below the support member 121.

Signal Processor 108

The signal processor 108 corrects sensitivity dispersion of the transducers 211 and the fixed transducer 103, and performs interpolation processing for the physically or electrically defective transducers for the photoacoustic wave digital signals generated by the signal receiver 107. Furthermore, the signal processor 108 can perform accumulating processing to reduce noise. The photoacoustic signal, which is acquired by detecting the photoacoustic wave emitted by the light absorbing substance inside the object 101 is normally a weak signal. By performing the accumulating and averaging processing on the photoacoustic signals repeatedly acquired at a same position from the object 101, the S/N of the photoacoustic signals can be improved while reducing the system noise.

Control Processor 109

The control processor 109 operates the operating system (OS) that controls and manages the basic resources of program operation, reads the program codes stored in the storage 114, and executes the functions described below. The control processor 109 also receives event notices that are generated by various operations (e.g. starting imaging) from the user via the operation unit 111, manages the object information acquiring operation, and controls each hardware via the system bus 110. The control processor 109 also controls the irradiation of light 131 and the position of the movable probe 102, which are required to generate the target object information.

Operation Unit 111

The operation unit 111 is an input apparatus to perform image processing operation on images, including setting parameters on imaging (e.g. visualization range of object information) and instructing the start of imaging. Normally the operation unit 111 is constituted by a mouse, keyboard, touch panel or the like, and notifies an event to software, such as the OS, running on the control processor 109 according to the operation by the user.

Image Constructing Unit 112

The image constructing unit 112 images the tissue information inside the object based on the acquired photoacoustic wave digital signals, and constructs a display image, such as an arbitrary tomographic image, of the photoacoustic wave image. The image constructing unit 112 also performs various correction processing operations, such as the correction of brightness and distortion, and the extraction of a target area, on the constructed image, so as to construct information more useful for diagnosis. The image constructing unit 112 also adjusts the parameters and display image to construct the photoacoustic wave image, according to the operation by the user via the operation unit 111. The photoacoustic wave image is acquired by performing image reconstruction processing for three-dimensional photoacoustic wave digital signals generated by the transducers 211 and fixed transducers 103, whereby object information, such as characteristic distribution of acoustic impedance and optical characteristic value distribution, can be visualized. For the image reconstruction processing, back projection in the time domain or Fourier domain, which is normally used in tomography technology, or phased addition processing, for example, is used. If the time constraints are not too strict, an image reconstruction method using repeated processing, such as inverse problem analysis, can be used, and instead of performing image reconstruction, object information may be visualized using a probe that includes a reception focus function of an acoustic lens or the like.

The image constructing unit 112 is normally constituted by a graphics processing unit (GPU) that includes a high performance computing function and a graphics display function. Thereby the time required for performing image reconstructing processing and constructing the display image can be decreased.

Display Unit 113

The display 113 displays the photoacoustic wave image constructed by the image constructing unit 112 and the UI for processing the image and operating the apparatus. A liquid crystal display, for example, may be used, but any type of display, such as an organic electro luminescence (EL) display, can be used.

Storage 114

The storage 114 is constituted by storage media, such as a memory required for the control processor 109 to operate, a memory that temporarily holds data during the object information acquiring operation, and a hard disk that stores and holds a generated photoacoustic wave image, and related object information and diagnosis information. The storage 114 also stores program codes of software to implement functions which are described later.

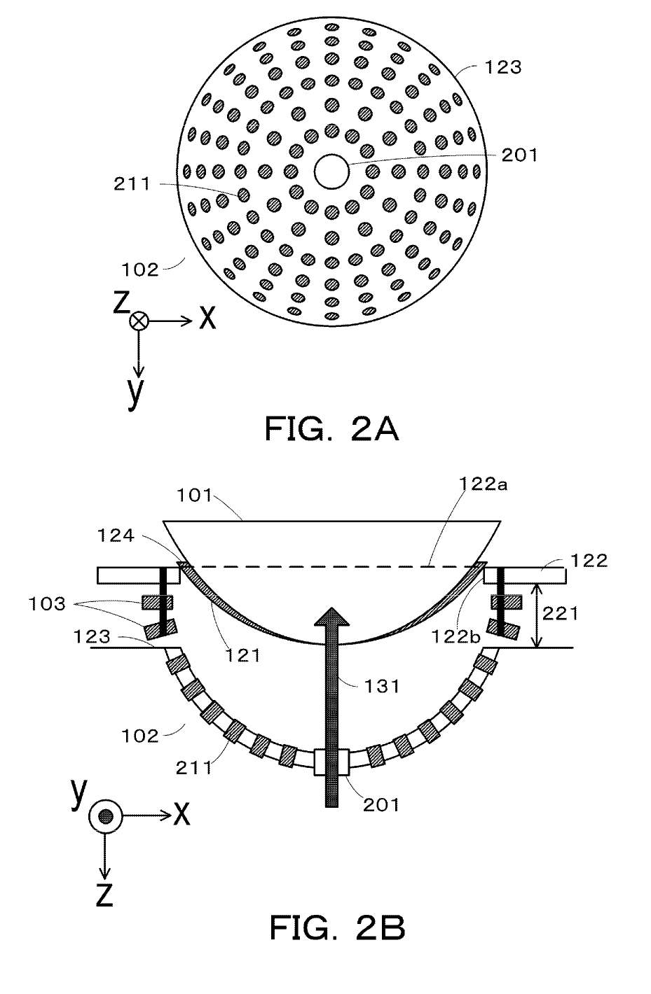

FIGS. 2A and 2B are conceptual diagrams depicting a configuration of the probe according to Example 1, where FIG. 2A is a plan view of the movable probe 102, and FIG. 2B is a cross-sectional view thereof. In other words, FIG. 2B is a cross-sectional view when the movable probe 102 in FIG. 2A is sectioned by a plane which passes through the center of the movable probe 102, and of which y axis direction is the normal direction. The movable probe 102 is constituted by a plurality of transducers which are arranged at different positions along an approximately spherical crown shape or bowl shape. In this example, it is assumed that the movable probe 102 includes a plurality of transducers 211. However the present invention is not limited to this, but the object information may be generated by movably controlling a probe group which has a support member that supports a plurality of probes along a spherical crown shape. The individual probes that are used in this case may be a probe where a plurality of transducers are disposed in a line, or may be an arrayed probe where a plurality of transducers are two-dimensionally arrayed.

These transducers 211 detect the photoacoustic wave which is generated inside the object 101 when the light 131 is irradiated onto the object 101, convert the photoacoustic wave into an electric signal, and transmit the electric signal as the photoacoustic wave signal, which is an analog signal.

The relative position of the movable probe 102, with respect to the object 101 or the opening 122b, is controlled by the position control mechanism 104. Therefore the movable probe 102 is disposed at a position where the object 101 and the support member 121 which supports the object 101 do not physically interfere with the movable probe 102 and the support member 123 thereof (breast support member) (bowl shape support member), according to the maximum range of the position control of the movable probe 102 with respect to the object 101. In other words, [the movable probe 102] is disposed in a position that is separated from the support member 122 (bed) by the distance 221. This means that in the normal direction (z direction) of the opening surface 122a of the opening 122b, which is a first opening into which the object 101 is inserted, the object 101 and the support member 121 thereof are separated from the movable probe 102 and the support member 123 thereof. The support member 122 may be a bed which supports a examinee having the object 101. The support member 121 is annually connected to the support member 122. The support member 121 is arranged to fill the opening 122b.

The movable probe 102 includes an irradiation port 201 to guide the light 131 to the bottom surface thereof, so that the light guided by the irradiation optical system 106 is irradiated onto the object 101 through the irradiation port 201. Here the light irradiator includes the irradiation port 201, the irradiation optical system 106 and the light source 105. The fixed transducer 103 is fixed to the support member 122 of the object 101. Further, as mentioned later, the fixed transducers 103 are disposed so as to orient to the area that can be visualized at high accuracy which is formed by the movable probe 102. Instead of the fixed transducers 103, a plurality of fixed probes, each of which is constituted by a plurality of transducers and is fixed to the support member 122, may be disposed. Instead of the light irradiation unit, an ultrasonic transmitter may be disposed, so that the ultrasonic wave is transmitted from the ultrasonic transmitter to the object 101, whereby an acoustic wave is propagated from the object 101 and the acoustic wave is received by each transducer 211 and 103.

In the present invention, any type can be used for the transducers 211 and the fixed transducers 103. For example, an transducer that uses piezoelectric ceramics (PZT), which is used for ultrasonic diagnosis apparatus (general object information acquiring apparatus), can be used. An electrostatic capacitance type Capacitive micro-machined ultrasonic transducer (CMUT) or a Magnetic MUT (MMUT) that uses a magnetic film may be used. Piezoelectric MUT (PMUT) that uses a piezoelectric thin film may also be used.

Further, it is preferable that the space between the support member 121 of the object 101 and the support member 123 of the movable probe 102, which is a propagation path of the photoacoustic wave, is filled with a medium having high acoustic wave propagation efficiency. The medium having high acoustic wave propagation efficiency is, for example, the acoustic matching liquid which is water or liquid which consists primarily of water. This space is also a propagation path of the light 131, hence this medium is transparent to the light 131, such as water. The space between the object 101 and the support member 121 is also a propagation path of the photoacoustic wave, therefore it is preferable to dispose water or an acoustic transfer medium 124 such as gel or a gel sheet for ultrasonic measurement in this space. The support member 121 has a front aspect and a back aspect in vertical direction (Z direction). The front aspect is on the gel. The back aspect is on the water. The support member 121 is an acoustic coupling member propagating the acoustic wave. The acoustic coupling member is, for example, polyethylene terephthalate resin.

In the apparatus 100 having this configuration, the photoacoustic signals of the object 101 can be measured using the movable probe 102 and the fixed transducers 103, and the target object information in a wide range can be acquired.

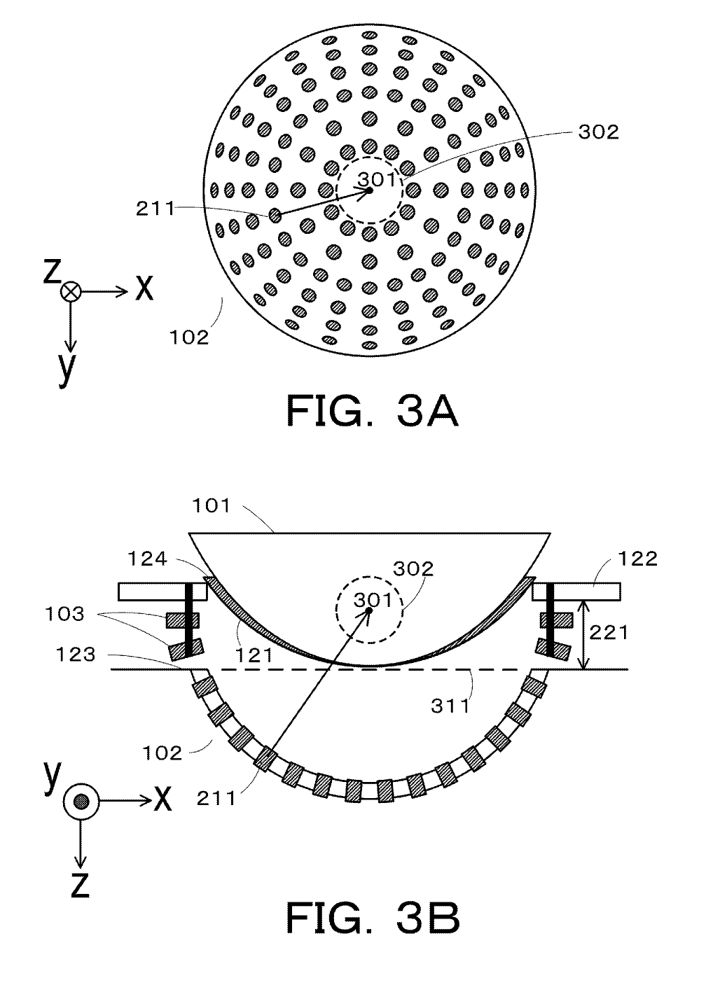

FIGS. 3A and 3B are conceptual diagrams depicting a reception characteristic of the probe according to Example 1, where FIG. 3A is a plan view of the movable probe 102, and FIG. 3B is a cross-sectional view thereof. In other words, FIG. 3B is a cross-sectional view when the movable probe 102 in FIG. 3A is sectioned by a plane which passes through the center of the movable probe 102, and of which y axis direction is the normal direction. The transducers 211 constituting the movable probe 102 are disposed along the approximately spherical crown shape, as depicted in FIGS. 2A and 2B. A curvature center point 301 indicates the curvature center point 301 here of the approximately spherical crown shape. In other words, the curvature center point 301 refers to the center of an approximately spherical shape that includes the approximately spherical crown shape. Normally an transducer has the highest reception sensitivity in the normal line direction of the reception surface thereof. Here the high sensitivity directions of the transducers 211 constituting the movable probe 102 are oriented to the curvature center point 301 of the approximately spherical crown shape. In other words, the plurality of transducers 211 are disposed such that the orientation axes of the acoustic wave detection elements concentrate in an area near the curvature center point 301. In other words, the plurality of transducers 211 are disposed such that the orientation axes of the acoustic wave detection elements overlap in an area near the curvature center point 301. Thereby an area 302 that can be visualized at high accuracy can be formed in the curvature center point 301 and its neighboring area. The orientation axis is a line constituted by a set of points located in the orientation direction of the acoustic wave detection element. In concrete terms, in FIG. 3B, the maximum value of the width of the area 302 (diameter of the area 302 in FIG. 3B) is on the support member 122, and is smaller than the width of the opening to which the object 101 is inserted. In other words, double the distance from the curvature center point 301 to the end of the area that can be visualized at high accuracy (portion indicated by the broken line in area 302 in FIG. 3B) is smaller than the width of the opening, to which the object 101 is inserted. The width of the area 302 and the shape of the area 302 can be adjusted by appropriately adjusting the arrangement of the transducers 211, the orientation directions or the like. Then object information in a wide range can be visualized at high accuracy by implementing scanning with the movable probe 102, that is, scanning the area 302 for the object using the position control mechanism 104.

As mentioned above, the movable probe 102 is disposed in a position that is separated from the support member 122 by the distance 221, so as not to interfere with the object 101 in the mechanical scanning. For example, if the depth of the support member 121 of the object 101 is 40 mm, the distance 221 can be larger than 40 mm, considering a slight deformation of the object 101 due to weighting. For this, the area 302 that can be visualized at high accuracy must be formed in such a way that the object 101 located in a higher area than the opening surface 311 of the movable probe 102 can be measured. As mentioned later, the area 302 that can be visualized at high accuracy is formed in a higher area than the opening surface 311, by limiting the solid angle of the approximately spherical crown shape of the movable probe 102. The surface drawn by the opening surface 311 by the movable probe 102, performing the later mentioned two-dimensional scanning, becomes the scanning surface.

In this example, the arrangement of the plurality of transducers 211 is not limited to the examples of the approximately spherical crown shape in FIG. 2A, 2B and FIG. 3A, 3B. It is sufficient if the direction having high reception sensitivity is oriented to a predetermined area, and a predetermined high accuracy area can be formed. In other words, the plurality of transducers may be disposed oriented to the predetermined area so that the area 302 that can be visualized at high accuracy can be formed. Further, the plurality of transducers may be disposed along a curved shape so as to satisfy this condition. It is sufficient if the high sensitivity directions of at least some of the plurality of transducers can intersect. It is also acceptable that no orientation direction of any of the plurality of transducers 211 intersects with the orientation directions of the other transducers 211, as long as all the orientation directions of the plurality of transducers 211 are directed toward the predetermined area 302. In this description, “curve” includes a true spherical shape and a spherical surface having an opening, such as a spherical crown shape. The curved surface also includes a surface that can be regarded as a spherical surface even if unevenness exists on the surface, and a surface on an elliptic sphere (a form generated by three-dimensionally extending an ellipse, of which surface is quadratic).

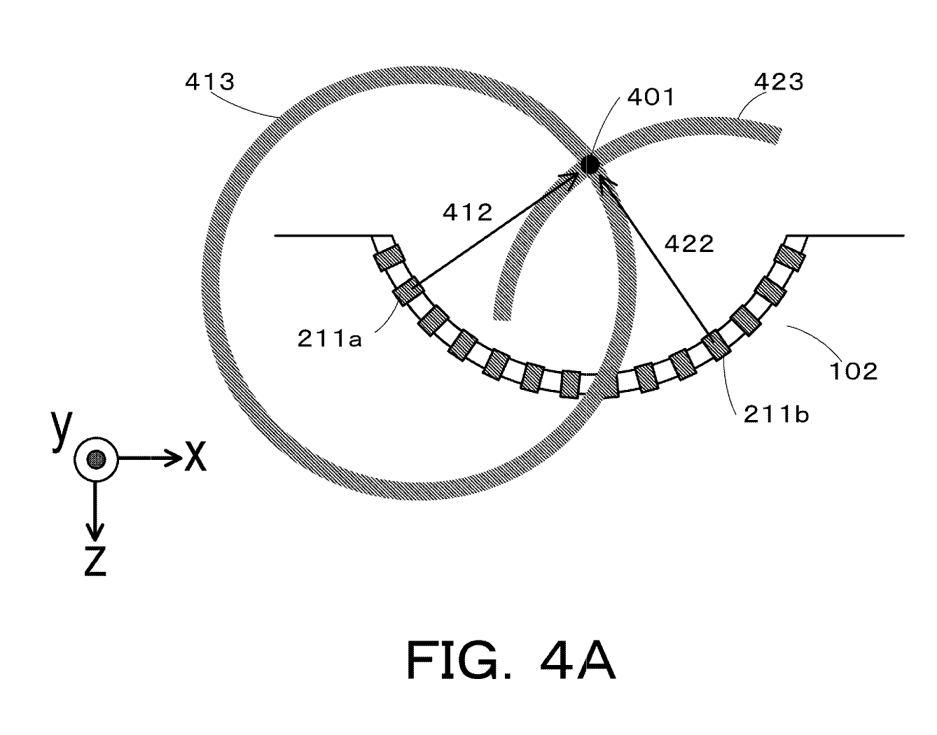

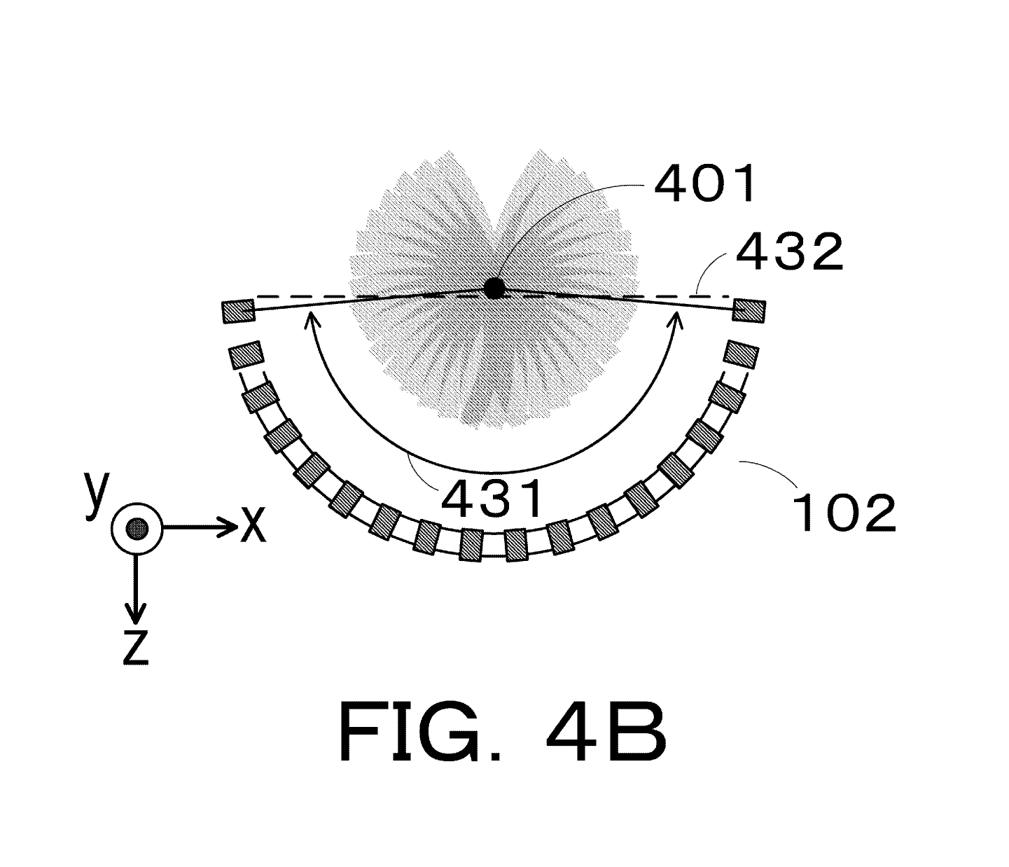

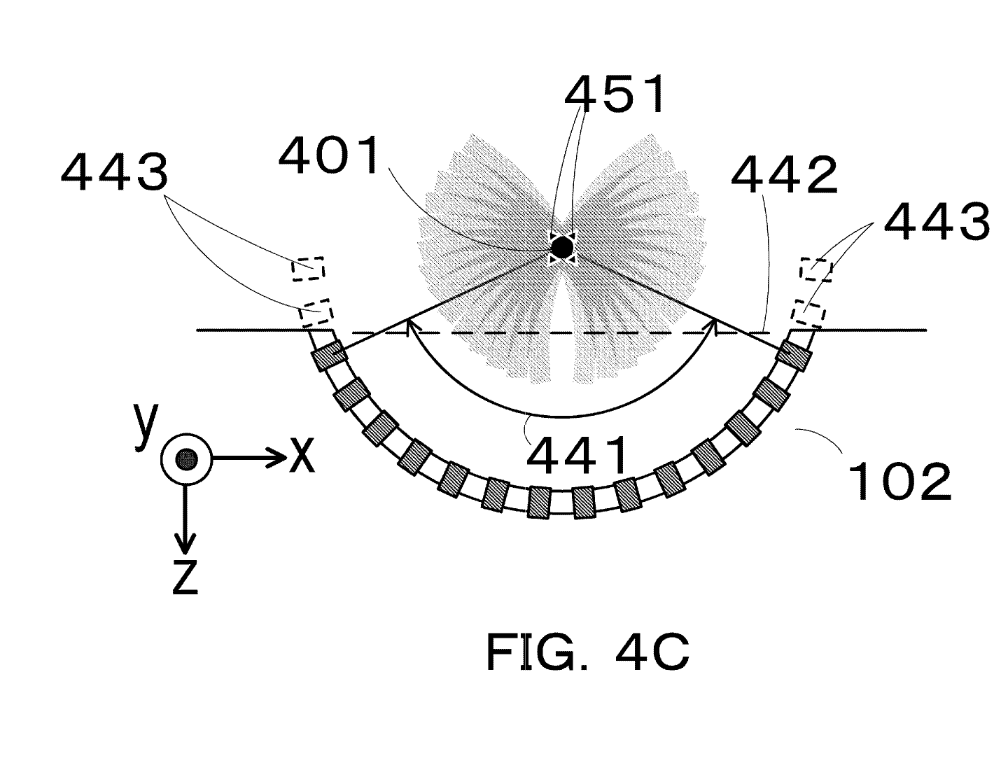

FIG. 4A is a diagram depicting an overview of the image reconstruction in Example 1. The overview of the image reconstruction will now be described with reference to FIG. 4A. A two-dimensional example will be described to simplify the explanation, but a three-dimensional example can be understood in the same manner. In this description, Circular Back Projection (CBP), which is one back projection method in a time domain, will be used. However, the present invention is not limited to this, but the Delay and Sum Method, the Hough Transform Algorithm (HTA) method or a back projection method in a Fourier domain may be used.

A sound source 401 is a spherical point sound source which is disposed in the curvature center point of the approximately spherical crown shape constituting the movable probe 102. In the CBP method, the position of the sound source 401 is estimated by back projecting a circle, of which radius is CT, where C denotes the sound velocity on the propagation path and T denotes the reception time of the acoustic wave, and of which center is a signal measurement point in real space (surface of the transducer). As illustrated in FIG. 4A, a circle 413, of which radius CT which is radius 412, is drawn from the transducer 211a. A circle (arc 423 is a part of this circle) with a radius CT is also drawn from the transducer 211b, which is disposed at a position that is different from the transducer 211a. Thereby the position of the sound source 401 can be more accurately estimated. In the same manner, circles with radius CT that are back-projected from all the measurement points are added, and as a result, the intersection thereof is imaged as a position of the sound source 401. In this example, to simplify explanation, the transducers including the transducer 211a are disposed along the approximately spherical crown shape, and the acoustic wave is irradiated from the sound source 401 located at the curvature center position. The present invention however is not limited to this, but back projection may be performed for transducers disposed on an arbitrary curved surface, along the propagation paths of the acoustic wave that radiate from the sound source, whereby the object can be visualized in the same manner.

FIG. 4B is a conceptual diagram depicting a tendency of an artifact generated by a probe having a semispherical shape. FIG. 4B shows the back-projection result of the position of the sound source 401 based on the CBP method when the solid angle 431 of the movable probe 102 is a solid angle in an ideal hemisphere closest to an approximately spherical crown shape. In an area near the sound source 401, back projection lines can be drawn from the sound source 401 in all directions, hence a three-dimensional position can be estimated at high accuracy. In other words, an artifact does not stand out around the sound source 401. In terms of suppressing an artifact, it is preferable to maximize the solid angle 431 of the movable probe 102. However, in the case of an approximately hemispherical surface of which solid angle 431 is large, the area that can be visualized at high accuracy is formed near the opening surface 432.

When the object information in a wide range is acquired by mechanically scanning the high accuracy area, it is possible to construct the movable probe 102 with a large diameter so as not to interfere with the object 101 during an entire scan. In this case however, the apparatus size increases as the diameter of the movable probe 102 increases, which leads to an increase in cost. Furthermore, the distance from the sound source 401 to the transducers increases, and efficiency to receive the source wave energy emitted by the sound source 401 drops, therefore the area of the receiving surface of the transducers must be increased.

FIG. 4C is a conceptual diagram depicting a tendency of an artifact generated in the probe according to Example 1. This probe 102 is the probe 102 of the present invention, but note that the present invention suppresses an artifact using this probe 102 and the fixed transducers 103, and does not suppress an artifact using the probe 102 in FIG. 4C alone.

FIG. 4C shows the back projection result based on the CBP method, for the approximately spherical crown shape having the solid angle 441, by which the area that can be visualized at high accuracy can be formed in a higher area than the opening surface 442. Compared with the solid angle 431, the solid angle 441 is limited. The back projection lines for the sound source 401 are polarized since the transducers 443, indicated by the broken line, cannot be disposed, that is, back projection from these orientations cannot be performed. An insufficiency in the orientations of the transducers 443 leads to a drop in the positional estimation accuracy of the sound source 401 in the directions indicated by the triangles 451, and as a result, an artifact indicated by the triangle 451 tends to stand out when an image is formed.

As described above, the movable probe 102 constituted by the solid angle 441 has a tendency to easily generate an artifact 451.

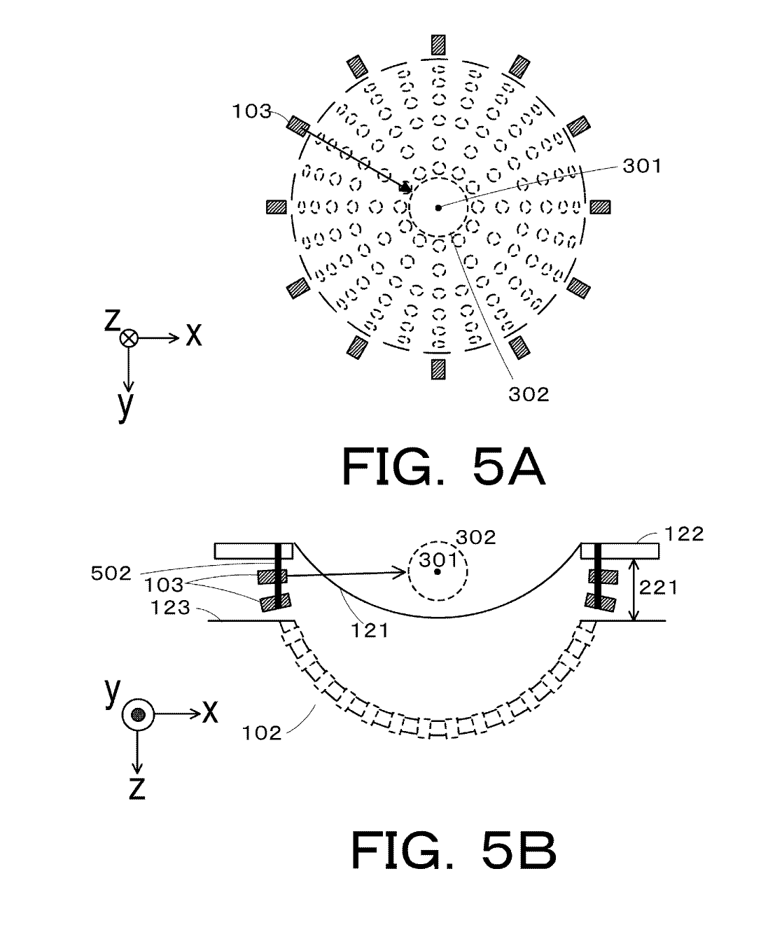

FIGS. 5A and 5B is a conceptual diagram depicting a configuration of the fixed transducers according to Example 1. FIG. 5A is a plan view of the movable probe 102, and FIG. 5B is a cross-sectional view thereof, that is, a cross-sectional view when the movable probe 102 in FIG. 5A is sectioned by a plane which passes through the center of the movable probe 102, and of which y axis direction is the normal direction. In the configuration of this example, a plurality of fixed transducers 103 are disposed, but the present invention is not limited to this, and a plurality of fixed probes, each of which has a plurality of transducers, may be disposed instead of the fixed transducers 103. In this case, an individual fixed probe may be a probe where a plurality of transducers are disposed in a line, or may be an arrayed probe where a plurality of transducers are two-dimensionally arrayed.

The fixed transducers 103 are disposed to compensate for missing transducers 443 in FIG. 4C, and are fixed to the support member 122 via a fixing member 502. The directions to which the fixed transducers 103 are oriented are directions towards the area 302 that can be visualized at high accuracy, which is formed by the movable probe 102. As shown in FIG. 5A, the opening to insert the object 101, disposed in the support member 122 as shown in FIG. 5A, is formed as a circle. But the shape of this opening may be different than a circle.

According to the above mentioned example, the distance 221 is at least 40 mm, hence it is more than possible to dispose the transducers. If liquid, such as water, is used as an acoustic transfer medium between the support member 121 that supports the object 101 and the movable probe 102, the fixed transducers 103 and the related electric wires (not illustrated) are water proofed and corrosion proofed.

Since the fixed transducers 103 are disposed to orient toward the area 302 that can be visualized at high accuracy, which is generated by the movable probe 102, the arrangement of the transducers along the approximately semispherical shape in FIG. 4B is reproduced. As a result, an artifact 451 shown in FIG. 4C which appears during imaging, can be suppressed, and the image quality of the area 302 can be more homogeneous.

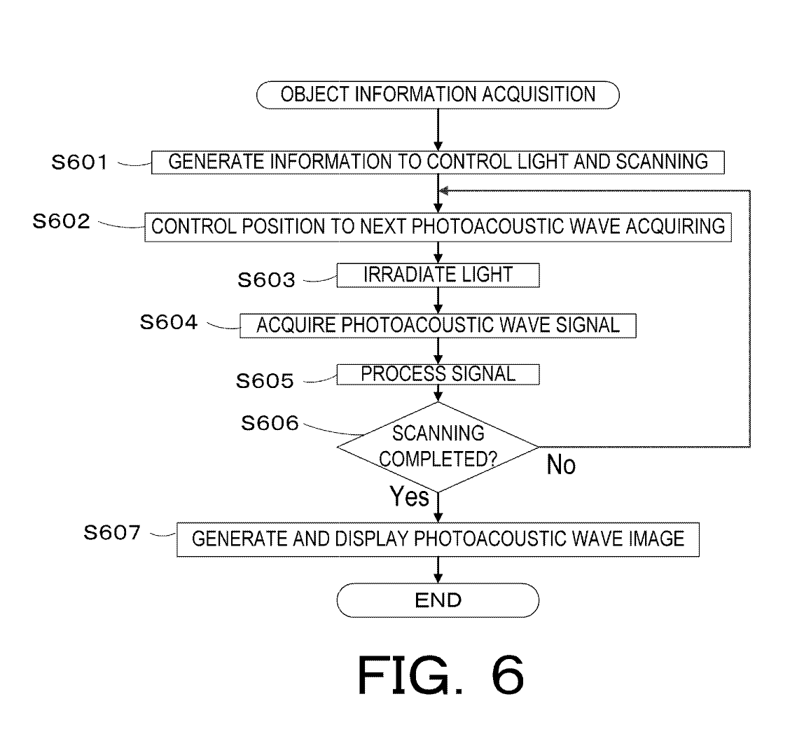

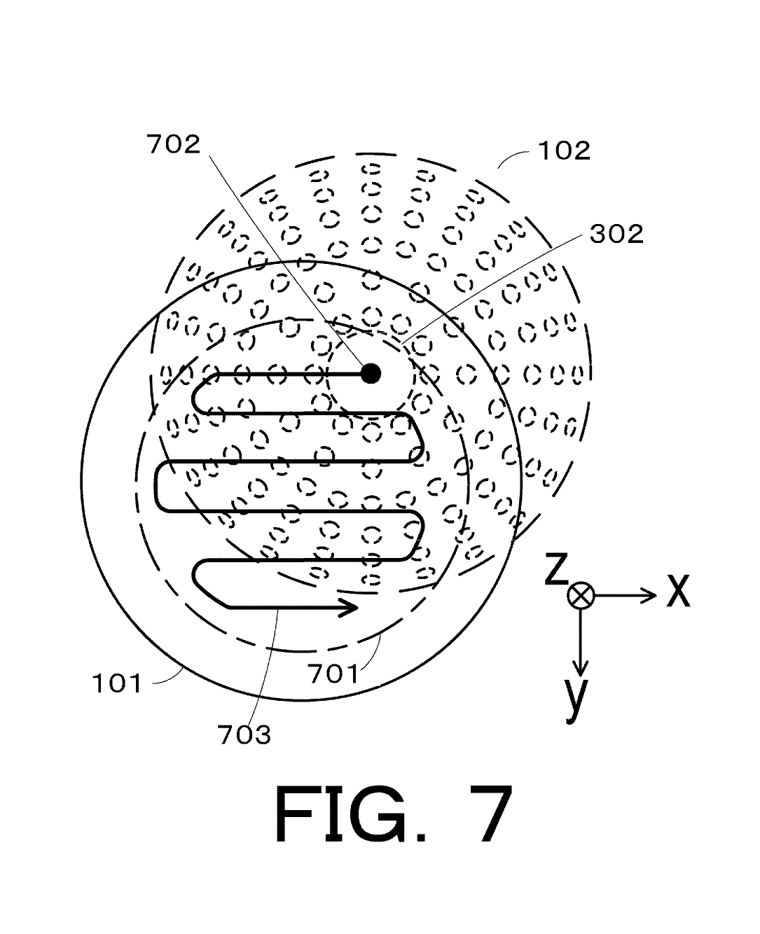

FIG. 6 is a flow chart depicting a flow of the object information acquisition according to Example 1. The flow is executed by the user instructing the start of imaging via the operation unit 111.

In step S601, the control processor 109 generates control information to control the following. In other words, the control processor 109 generates control information, such as information to control the later mentioned speed of two-dimensional scanning and scanning density of the movable probe 102, and a number of times of irradiation of the light 131, according to the imaging range of the object information, which the user specified via the operation unit 111, and the parameters required for generating the target object information. The control processor 109 outputs this control information to the position control mechanism 104, the light source 105 and the signal receiver 107. In step S602, the position control mechanism 104 performs position control to move the movable probe 102 to a position to acquire the next photoacoustic wave signal according to the scanning control information. In step S603, the light source 105 emits the pulsed light according to the emission start instruction from the control processor 109. The pulsed light emitted from the light source 105 is shaped to the light 131 via the irradiation optical system 106, and is irradiated onto the object 101. The irradiation optical system 106 generates a synchronization signal simultaneously with the irradiation of the light 131 onto the object 101, and transmits the synchronization signal to the position control mechanism 104 and the signal receiver 107.

In step S604, the movable probe 102 and the fixed transducers 103 detect the photoacoustic wave generated as a result of irradiating the light 131 onto the object 101, and transmit the analog photoacoustic signal. Then the signal receiver 107 starts receiving the photoacoustic wave signal synchronizing with the synchronization signal inputted from the irradiation optical system 106, and converts the analog photoacoustic signal into the photoacoustic wave digital signal. From this timing, the signal receiver 107 that received the synchronization signal starts receiving the photoacoustic wave signal for a predetermined number of samples at a predetermined sampling rate. The number of samples is determined considering the acoustic wave propagation speed in the object 101 and the maximum measurement depth as the apparatus specifications. The position control mechanism 104 transfers the position control information at the irradiation of the light 131 to the control processor 109 synchronizing with the synchronization signal that is inputted from the irradiation optical system 106.

In step S605, the signal processor 108 corrects sensitivity dispersion for each transducer, and performs interpolation processing for physically or electrically missing transducers for the photoacoustic wave digital signal based on the photoacoustic wave signals acquired by the fixed transducers 103 and the movable probe 102 respectively. The signal processor 108 also performs accumulating processing to reduce noise, for the photoacoustic wave signals outputted from the fixed transducers 103 of which positions are not controlled, and of which relative positions with respect to the object 101 are constant. The control processor 109 associates the photoacoustic wave digital signal, to which the signal processor 108 has applied various processing operations, with the position control information before storing this associated signal in the storage 114. In step S606, it is determined whether all the scanning required for generating the object information in the later mentioned imaging range 701 is completed. If all the scanning is not completed (S606: NO), processing moves to step S602, and the acquisition of the photoacoustic wave signal is repeated. If all the scanning is completed (S606: YES), then processing advances to step S607.

In step S607, the image constructing unit 112 starts the generation of the photoacoustic image, that is, image reconstruction, using the photoacoustic wave digital signals stored in the previous steps and the position control information when these signals were acquired. Normally image reconstruction processing takes time, and a GPU could perform this processing, therefore the image reconstruction processing may be executed in parallel with the signal acquisition operation. Then the display 113 displays the photoacoustic wave image generated by the image constructing unit 112, that is, the volume data of the object information in a display format required for diagnosis.

In the example in FIG. 6, using the characteristics of the fixed transducers 103, of which relative positions with respect to the object 101 are always constant, accumulating processing to reduce noise is performed to photoacoustic wave digital signals acquired by the fixed transducers 103. Therefore the photoacoustic wave digital signals acquired by the movable probe 102 and the fixed transducers 103 respectively are individually reconstructed into an image, and are synthesized with the volume data of the object information. However, the present invention is not limited to this, and the photoacoustic wave digital signals acquired from all the transducers of the movable probe 102 and the fixed transducers 103, acquired by one light irradiation, may be reconstructed into an image all at once.

FIG. 7 is a conceptual diagram depicting two-dimensional scanning control of the probe according to Example 1, and is a top view of the object 101 and the movable probe 102. An imaging range 701 indicates the imaging range specified by the user. A center point 702 is a center point of the area 320 that can be visualized at high accuracy by the movable probe 102, and a scanning locus 703 indicates the scanning locus of the center point 702 of the high accuracy area. The area 302 includes the center point 702 and is formed near the center point 702.

The scanning locus 703 is a locus of two-dimensional scanning that is required for acquiring the object information of the imaging range 701 in the area 302 that can be visualized at high accuracy. The control processor 109 generates the control information for the two-dimensional scanning according to the scanning locus 703, and controls the position control mechanism 104. The scanning control of the movable probe 102 according to the present invention is not limited to the example in FIG. 7. A two-dimensional spiral locus or a locus combining a plurality of linear loci may also be used. For the position control of the movable probe 102, the movable probe 102 may be continuously moved on the scanning locus 703, or may stop at each point (each signal acquiring position) on the locus 703 and move to the next point (step and repeat method). Furthermore, scanning control may be three-dimensional (in XYZ space), not only a two-dimensional (in XY plane) movement. In concrete terms, the position control mechanism 104 may be constructed so that the area 302, that can be visualized at high accuracy can be scanned in the depth direction (Z direction) of the object 101, in other words, the position of the movable probe 102 can be controlled vertically.

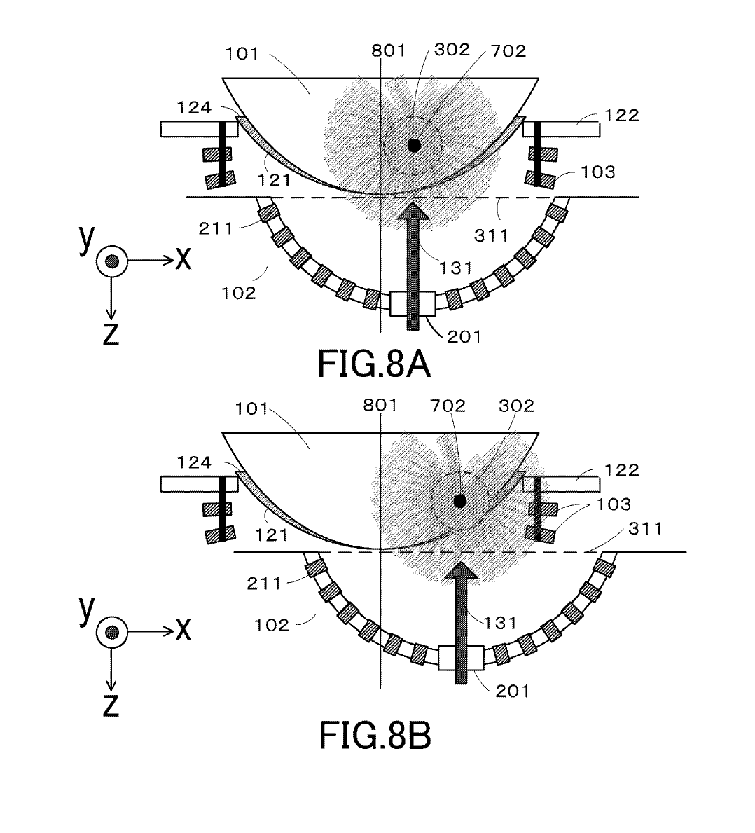

FIGS. 8A and 8B are conceptual diagrams depicting the artifact suppression effect according to Example 1. FIG. 8A and FIG. 8B are both cross-sectional views when the movable probe 102, the fixed transducers 103, the object 101 and the neighborhood area thereof are sectioned by a plane of which y axis direction is the normal direction thereof. Further, each back projection state of all the transducers of the movable probe 102 and the fixed transducers 103, at different positions with respect to the center axis 801 of the object 101 based on the CBP method, is also illustrated. The positional relationship between the movable probe 102 and the fixed transducers 103 changes due to the position control by the movable probe 102. However, both FIG. 8A and FIG. 8B show that the back projection line can be drawn in the missing directions if the movable probe 102 exists when viewing from the center point 702 of the high accuracy area of the movable probe 102, and accuracy to estimate the three-dimensional position can be improved. As the location becomes more distant from the center axis of the object 101, back projection tends to be slightly polarized, as shown in FIG. 8B, but the fixed transducers 103 can compensate for the missing directions which the movable probe 102 cannot cover.

By disposing the fixed transducers 103 like this, the solid angle which becomes insufficient by the movable probe 102 alone, can always be compensated for, regardless the position of the movable probe 102.

According to the apparatus 100 having the above configuration, the photoacoustic wave is acquired by the movable probe 102 constituted by a plurality of transducers disposed in different positions on the curved surface and the fixed transducers 103 disposed to compensate for the solid angle of the movable probe 102. Thereby an artifact which stands out when imaging the object information can be reduced.

Moreover, the artifact suppression effect can also be demonstrated in the arbitrary position control by the movable probe 102. As a result, an image of the object information can be generated at high accuracy, homogeneously, and in a wide range.

### Example 2

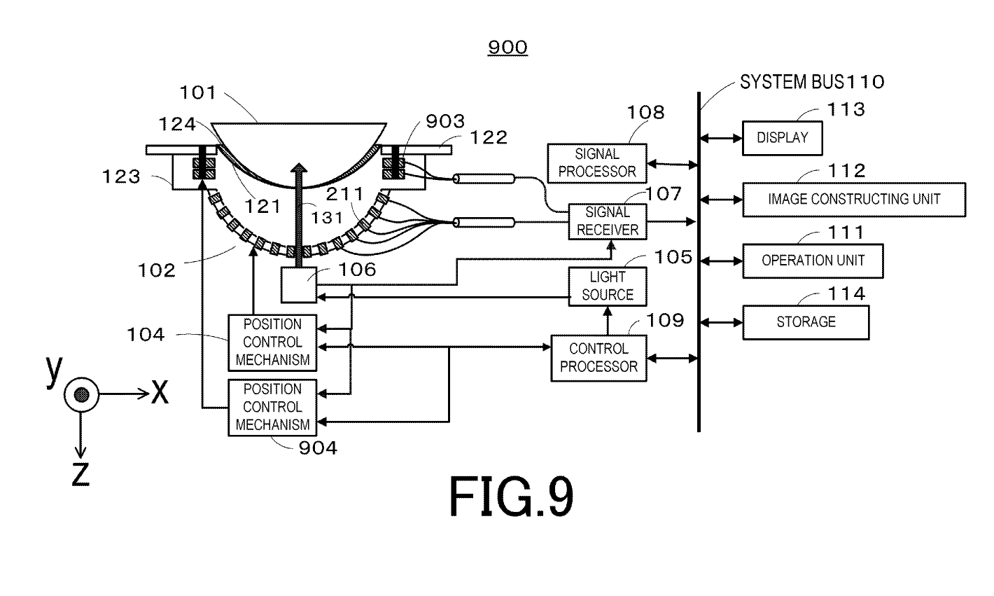

FIG. 9 is a schematic diagram depicting Example 2 of an object information acquiring apparatus 900 (hereafter called “apparatus 900”) of the present invention, where a same composing element as Example 1 is denoted with a same reference numeral, and redundant description thereof is omitted. In Example 1 described above, the fixed transducers 103, which are fixed to the support member 122 of the apparatus 100, are used to compensate for the solid angle of the movable probe 102. In this example, in addition to the two-dimensional scanning control by the movable probe 102, the positions of the fixed transducers 903 are controlled. Primarily the characteristic part of this example will be described herein below.

In addition to the configuration of the apparatus 100 illustrated in FIG. 1, the apparatus 900 includes fixed transducers 903 of which positions are controllable, and a position control mechanism 904 that controls the positions of the fixed transducers 903. The position control mechanism 904 is constituted by a drive member, such as a motor, and mechanical parts that transfer the driving force thereof, just like the position control mechanism 104. The position control mechanism 904 further includes a mechanism which can control the positions of the fixed transducers 903 in the circumferential direction along the circular opening of the support member 122, through which the object 101 is inserted. Moreover, the position control mechanism 904 outputs the current position control information to the control processor 109, synchronizing with each emission control of the light 131 by the irradiation optical system 106. In this configuration, the position control mechanism 104 and the position control mechanism 904 are separate hardware, but respective functions may be integrated into one hardware configuration.

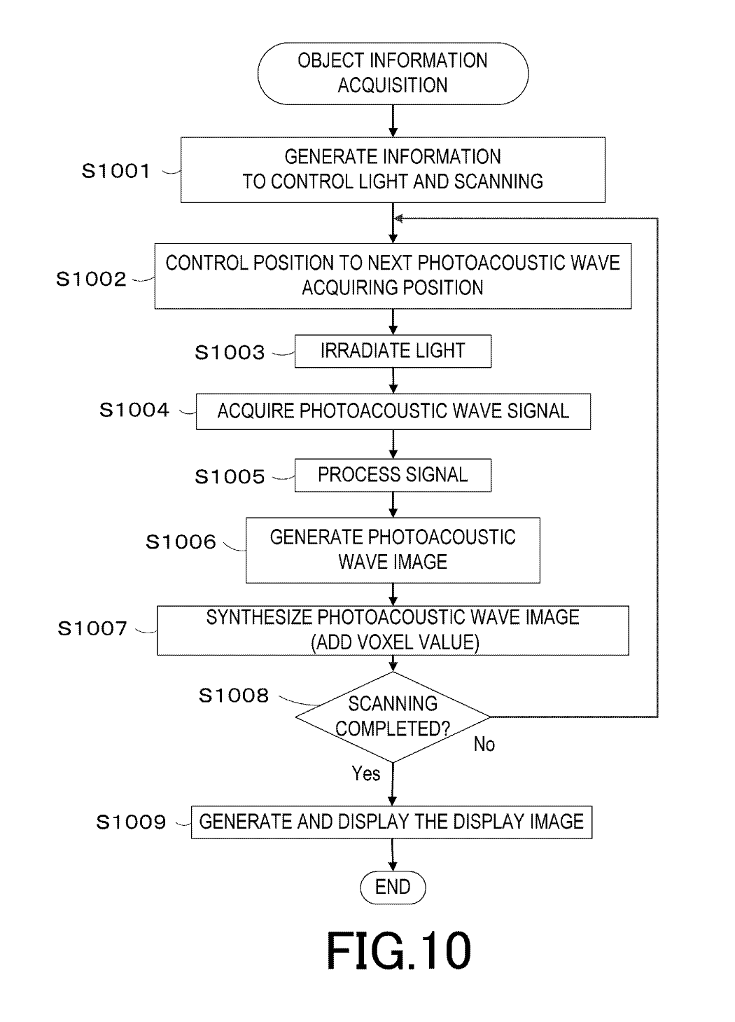

FIG. 10 is a flow chart depicting a flow of the object information acquisition according to Example 2. The flow starts when the user instructs for the start of imaging via the operation unit 111. In step S1001, the control processor 109 generates control information to control the following. In other words, the control processor 109 generates control information, such as information to control the scanning speed and the scanning density of the movable probe 102, the latter described scanning speed and scanning density of the fixed transducers 903, and a number of times of irradiation of the light 131. This information is generated according to the imaging range of the object information which the user specified via the operation unit 111 and the parameters required for generating the target object information. Further, the control processor 109 outputs this control information to the position control mechanisms 104 and 904, the light source 105 and the signal receiver 107. In step S1002, the position control mechanisms 104 and 904 perform position control to move the positions of the movable probe 102 and the fixed transducers 903 to positions to acquire the next photoacoustic wave signal according to the scanning control information.

In step S1003, the light source 105 emits the pulsed light according to the emission start instruction from the control processor 109. The pulse light emitted from the light source 105 is shaped to the light 131 via the irradiation optical system 106, and is irradiated onto the object 101. The irradiation optical system 106 generates a synchronization signal simultaneously with the irradiation of the light 131 to the object 101, and transmits the synchronization signal to the position control mechanism 104 and 904, and the signal receiver 107.

In step S1004, the movable probe 102 and the fixed transducers 103 detect the photoacoustic wave generated as a result of irradiating the light 131 onto the object 101, and transmit the analog photoacoustic signals to the signal receiver 107. The signal receiver 107 starts receiving the photoacoustic wave signals synchronizing with the synchronization signal inputted from the irradiation optical system 106, and converts the analog photoacoustic signals into digital signals, and outputs the photoacoustic wave digital signals. From this timing, the signal receiver 107 that received the synchronization signal starts receiving the photoacoustic wave signals for a predetermined number of samples at a predetermined sampling rate. The position control mechanisms 104 and 904 transfer the position control information of the movable probe 102 and the fixed transducers 903, which was acquired when the light 131 was irradiated, to the control processor 109 respectively, synchronizing with the synchronization signal inputted from the irradiation optical system 106.

In step S1005, the signal processor 108 corrects sensitivity dispersion for each transducer, for the photoacoustic wave digital signals constituted by the photoacoustic waves acquired by the movable probe 102 and the fixed transducers 903 respectively. The signal processor 108 also performs interpolation processing for the physically or electrically missing transducers. In this example, the signals acquired from all the transducers 211 of the movable probe 102 and the fixed transducers 903, acquired by one light irradiation, may be collectively regarded as one photoacoustic signal. In step S1006, the image constructing unit 112 starts generation of the photoacoustic image, using the photoacoustic wave digital signals acquired by step S1005 and the position control information when these signals were acquired. As mentioned above, generation of the photoacoustic wave image may be executed in parallel with the signal acquisition operation. If the image reconstruction processing delays behind the repeat cycle of acquiring the photoacoustic wave signals, the photoacoustic wave digital signals acquired in succession may be managed in a queue as signal data, then the image constructing unit 112 can sequentially image the signal data added to the queue.

In step S1007, the image constructing unit 112 adds the photoacoustic wave image generated in step S1006 to the voxel value considering the position on the volume data of the object information that is finally generated. In step S1008, it is determined whether all the scanning required for generating the target object information is completed. If all the scanning is not completed, processing moves to step S1001, and acquisition of the photoacoustic wave signal is repeated. If all the scanning is completed, then processing advances to step S1009. In step S1009, the display 113 displays the volume data of the object information generated by the image constructing unit 112 in a display format required for diagnosis.

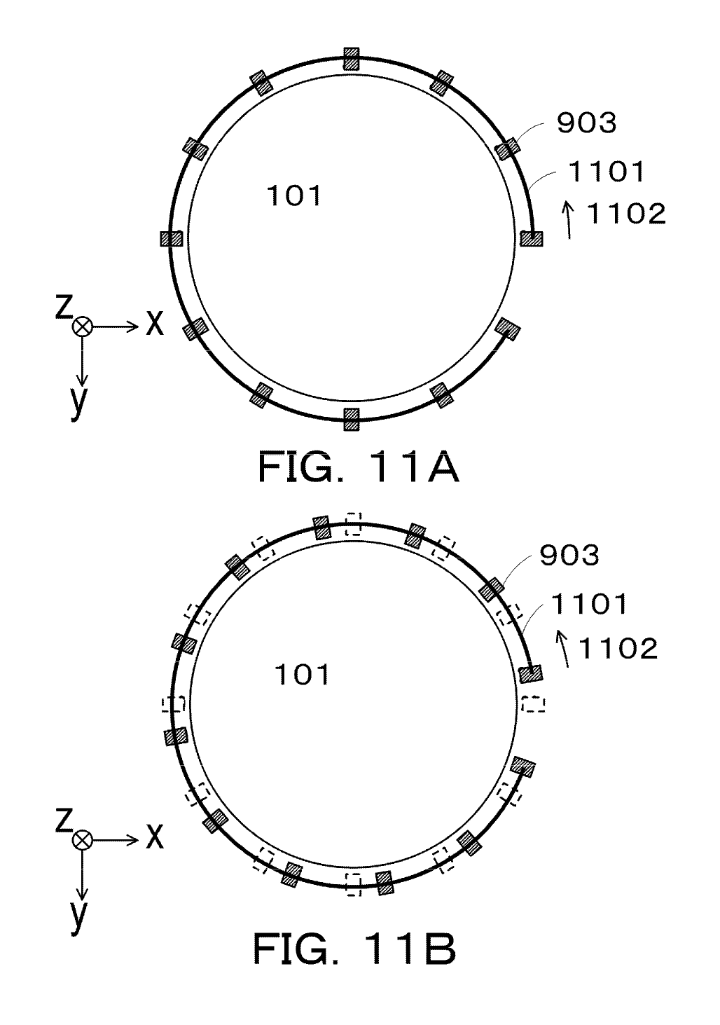

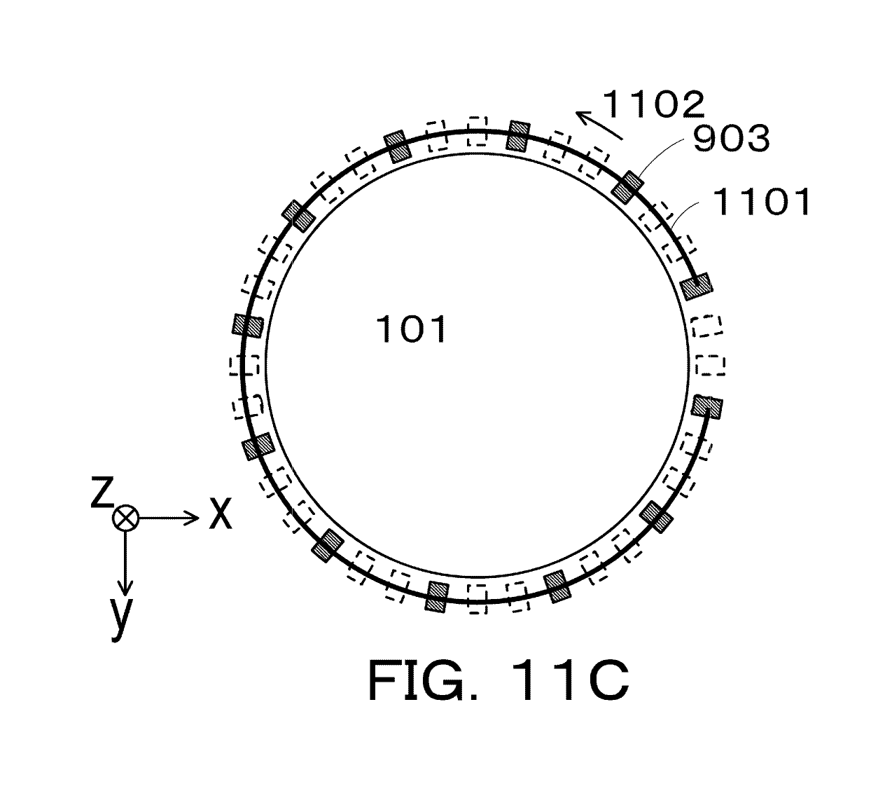

FIGS. 11A, 11B, and 11C is a conceptual diagram depicting position control of the fixed transducers in Example 2. FIG. 11A to FIG. 11C are top view of the fixed transducers 903. The position control mechanism 904 mentioned above is a rail 1101, for example, for controlling the positions of the fixed transducers 903 in the circumferential direction of the object 101. The fixed transducers 903 are disposed on the rail 1101, for example, and the positions of all the fixed transducers 903 are controlled all at once.

To control the positions of the fixed transducers 903, position control is started at the positions in FIG. 11A when the object information acquisition is started, and the positions are controlled as in FIG. 11B to FIG. 11C at a constant angular velocity. By this position control, an apparent number of measurement points can be increased, and the artifact suppression effect on the xy plane can be further demonstrated. Even if a number of fixed transducers 903 is small, the effect of reducing artifacts can be demonstrated by performing this position control.

One positional control process in FIG. 11A to FIG. 11C may be performed in accordance with the scanning time of the movable probe 102, or reciprocating control of the process in FIG. 11A to FIG. 11C may be repeated. The present invention is not limited to this, but positions of fixed probes having a plurality of transducers, instead of fixed transducers 903, may be controlled in a circumferential direction of the object, in other words, each fixed probe is disposed on the rail 1101, so that the positions of all the fixed probes are controlled all at once. Each probe that is used in this case may be a probe where a plurality of transducers are disposed in a line, or may be an arrayed probe where a plurality of transducers are two-dimensionally arrayed.

According to the apparatus 900 having this configuration, position control is performed not only for the movable probe 102 constituted by a plurality of transducers 211 disposed on different positions on the curves surface, but also for the fixed transducers 903 disposed to compensate for the solid angle of the movable probe 102. Thereby an artifact which stands out when imaging the acquired object information can be suppressed. Furthermore, even if a number of fixed transducers 903 is small, a same effect as the case of including many fixed transducers can be demonstrated by the positional control. This is also true for the vibrators 211.

In other words, an artifact can be suppressed by scanning with further including the position control mechanism 904 that can control the positions of the fixed transducers 903 along the circumferential direction of the opening through which the object 101 is inserted.

### Example 3

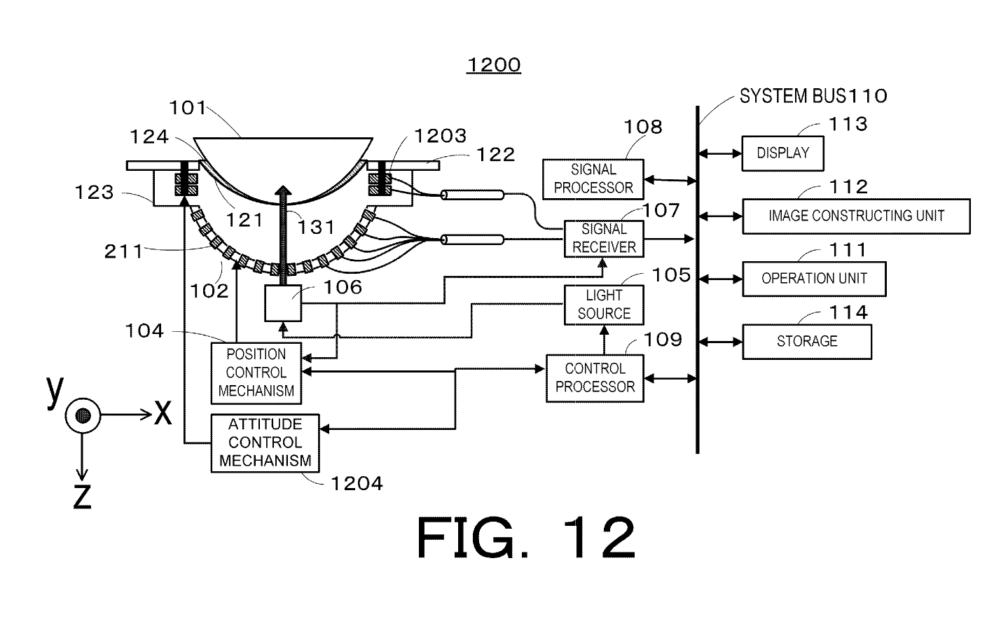

FIG. 12 is a schematic diagram depicting Example 3 of an object information acquiring apparatus 1200 (hereafter called “apparatus 1200”) of the present invention. In Example 2, the positions of the fixed transducers 903 are also controlled, whereby the artifact suppression effect is demonstrated. In Example 3, attitude control is performed for the fixed transducers 1203, in addition to the two-dimensional scanning control of the movable probe 102. Primarily the characteristic part of this example will be described herein below.

Instead of the mobile probe 903 and the position control mechanism 904 in the configuration of the apparatus 900 illustrated in FIG. 9, the apparatus 1200 includes fixed transducers 1203 of the present invention and an attitude control mechanism 1204, which is an attitude controller that controls the orientation directions of the fixed transducers 1203. Just like the position control mechanism 104, the attitude control mechanism 1204 is constituted by a drive member, such as a motor, and mechanical parts that transfer the driving force thereof. Further, the attitude control mechanism 1204 includes a mechanism that can control the orientation directions of a plurality of transducers constituting the fixed transducers 1203. Furthermore, the attitude control mechanism 1204 outputs the current attitude control information to the control processor 109 synchronizing with each emission control of the light 131 by the irradiation optical system 106. The position control mechanism 104 and the attitude control mechanism 1204 are separate hardware, but the respective functions may be integrated into a same hardware configuration. For the attitude control of the fixed transducers 1203 in this example, the orientation direction of each transducer may be controlled so as to always orient to the area 302, that can be visualized at high accuracy, which is generated by the moving probe 102, without depending on the position control of the movable probe 102.

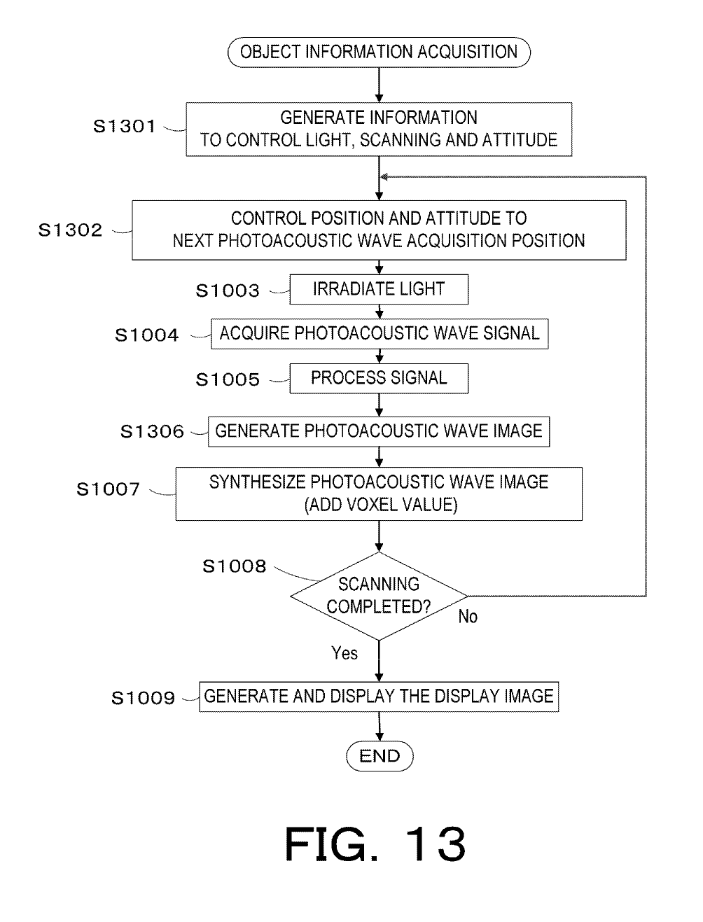

FIG. 13 is a flow chart depicting a flow of the object information acquisition according to Example 3. Instead of steps S1001, S1002 and S1006 in the flow shown in FIG. 10, Example 3 includes steps S1301, S1302 and S1306, and only the characteristic steps of this example will be described here. The rest of the processing is the same as FIG. 10.

In step S1301, the control processor 109 generates control information, including scanning information, such as the scanning speed and the scanning density of the movable probe 102, the later described attitude information of the fixed transducers 1203, and a number of times of irradiating the light 131. At this time, the control information is generated in accordance with the imaging range of the object information, parameters required for generating the target object information or the like, which the user specified via the operation unit 111. Furthermore, the control processor 109 outputs the control information to the position control mechanism 104, the attitude control mechanism 1204, the light source 105 and the signal receiver 107.

In step S1302, the position control mechanism 104 performs position control to move the position of the movable probe 102 to the next photoacoustic signal acquisition position according to the scanning control information. In step S1306, the image constructing unit 112 starts generating the photoacoustic wave image using the photoacoustic digital signals acquired in the previous steps, the position control information of the movable probe 102 when this signal was acquired, and the later described attitude control information of the fixed operators 1203. The rest of the processing operations are the same as the flow depicted in FIG. 10.

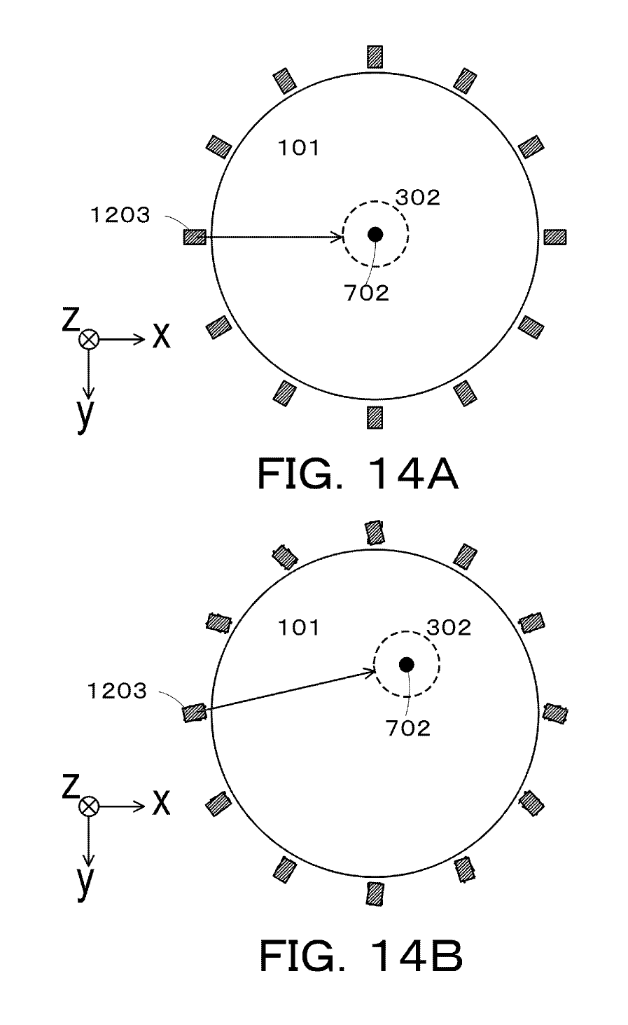

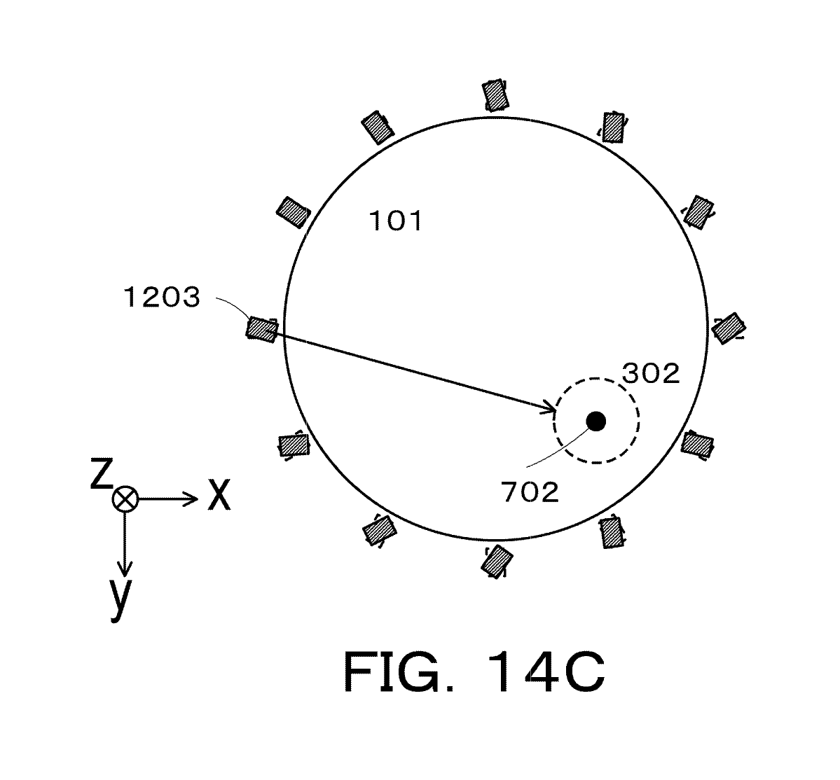

FIGS. 14A, 14B, and 14C are conceptual drawings depicting the attitude control of the rotation angles of the fixed transducers 1203 according to Example 3. FIG. 14A to FIG. 14C are top views of the fixed transducers 1203, where the state of the rotation angle control in the xy plane direction with the z axis as the shaft is shown. The present invention is not limited to this, but may have a configuration of a plurality of fixed probes, instead of the fixed transducers 1203, which are disposed in the circumferential direction of the object 101, where each of the fixed probes has a plurality of transducers so that the fixed probes are controlled in the xy plane direction with the z axis as the shaft. The individual fixed probe used in this case may be a probe where a plurality of transducers are disposed in a line, or an arrayed probe where a plurality of transducers are two-dimensionally arrayed.

FIG. 14A shows a case when the area 302 that can be visualized at high accuracy, which is formed by the movable probe 102, and the center point 702 thereof, are located at the center of the object 101 or the support member 121 thereof. In this case, the attitude control mechanism 1204 controls the rotation angle so that the fixed transducers 1203 orient to the center portion of the object 101 or the support member 121 thereof. If the high accuracy area 302 is moved from the center of the object 101 by the position control of the movable probe 102, the attitude control mechanism 1204 controls the orientation directions of the transducers 1203 as shown in FIG. 14B, so as to maintain the orientation to the high accuracy area 302, as illustrated in FIG. 14B. FIG. 14C shows an example when the movable probe 102 receives the position control. As illustrated in FIG. 14A to FIG. 14C, the attitude control mechanism 1204 controls the attitude of the fixed transducers 1203. Thereby the orientation of the fixed transducers 1203 can be maintained to the direction to the high accuracy area 302, even if the positional relationship between the movable probe 102 and the fixed transducers 1203 is changed by the position control of the movable probe 102.

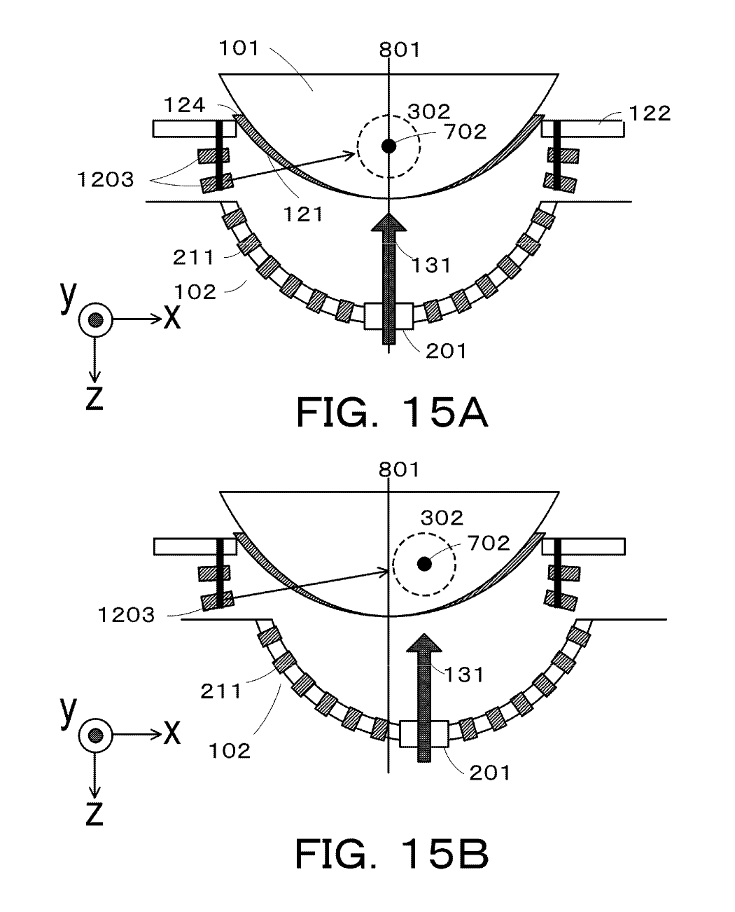

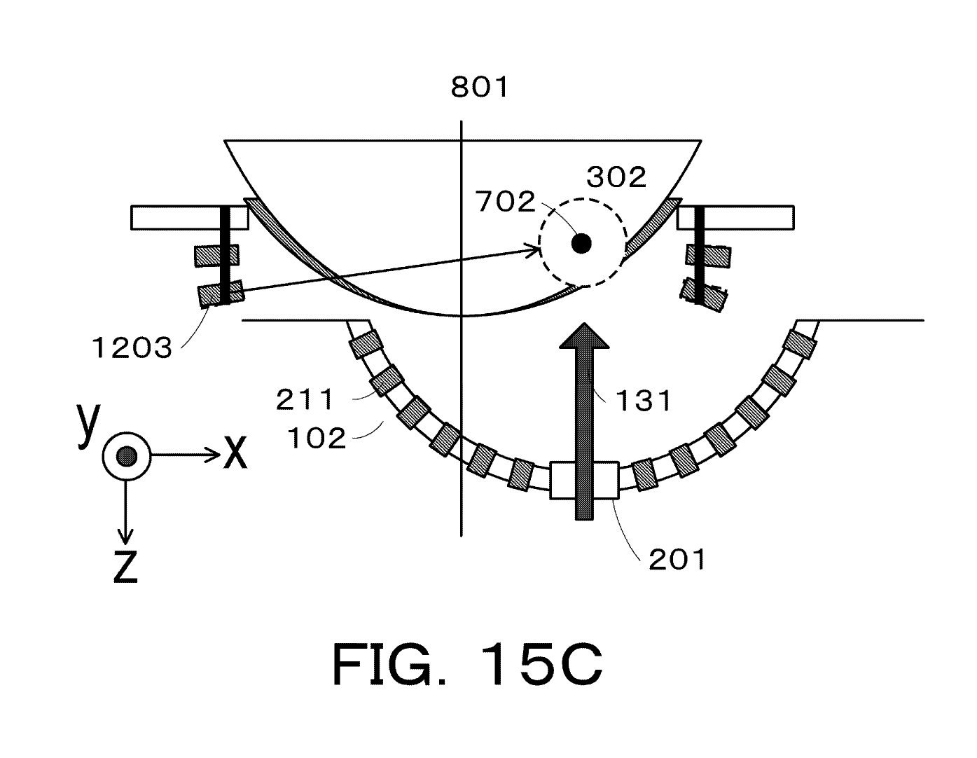

FIGS. 15A, 15B, and 15C are conceptual diagrams depicting the attitude control of the elevation/depression angles of the fixed transducers according to Example 3. FIG. 15A to FIG. 15C are cross-sectional views of the fixed transducers 1203, and show a state of the elevation/depression angle control for the transducers 1401. FIG. 15A shows a case when the area 302, that can be visualized at high accuracy, which is formed by the movable probe 102, and the center point thereof 702, are located on the center axis 801 of the object 101 or the support member 121 thereof. In this case, the attitude control mechanism 1204 controls the fixed transducers 1203 to have an elevation/depression angle such that the fixed transducers 1203 are disposed on an extended line of the approximately spherical crown shape of the movable probe 102, that is, along the approximately spherical shape. FIG. 15B and FIG. 15C show a state of the elevation/depression angle control, that is, a state of attitude control on the XZ plane, when the center point 702 moves away from the center axis 801 of the object 101 respectively. As illustrated in FIG. 15B and FIG. 15C, the attitude of each fixed transducer 1203 is controlled so as to decrease the elevation/depression angle of the fixed transducers 1203 that moves away from the area 302 that can be visualized at high accuracy, and increase the elevation/depression angle of the fixed transducer 1203 that moves toward the high accuracy area 302. By this attitude control, the orientation directions of the fixed transducers 1203 can be maintained toward the high accuracy area 302, even if the positional relationship between the movable probe 102 and the fixed transducers 1203 is changed by the position control of the movable probe 102. In other words, the orientation directions of the fixed transducers 1203 can be maintained toward the high accuracy area 302 by changing the orientation directions of the fixed transducers 1203 to follow up the position of the high accuracy area 302, even if the position is changed by the change of the movable probe 102. This means that the attitude control mechanism 1204 performs the attitude control of the fixed transducers 1203 in the X, Y and Z directions according to the attitude control information illustrated in FIG. 14A to 14C and FIG. 15A to 15C.

In FIGS. 14 A to 14C and FIGS. 15 A to 15C, it is assumed that the position control of the movable probe 102 is by two-dimensional scanning on the xy plane, in order to simplify explanation. The present invention is not limited to this, and even in the case when the position control mechanism 104 includes a configuration to control the position of the movable probe 102 in the vertical direction (Z direction), whereby three-dimensional scanning control can be performed, the attitude control of the fixed transducers 1203 can be performed with following up the three-dimensional scanning in the same manner. Instead of following up the position control of the movable probe 102, the position control of the movable probe 102 and the attitude control of the fixed transducers 1203 may be independently controlled, so as to orient to the same high accuracy area 302.

The apparatus 1200 having this configuration controls the position of the movable probe 102 constituted by a plurality of transducers 211 disposed at different positions on a curved surface, and also controls the attitude of the fixed transducers 1203. Thereby the artifact suppression effect can be highly maintained, even when controlling the position of the movable probe 102 in a wide range. As a result, the object information that is highly accurate and more homogeneous can be acquired in a wide range.

### Example 4

The object of the present invention is achieved as follows. A storage medium (or recording medium) storing program codes of software, to implement the functions of the above mentioned examples, is supplied to a system or apparatus. Then a computer (or CPU or MPU) of the system or apparatus reads and executes the program codes stored in the storage medium. In this case, the program codes read from the storage medium implement the functions of the above mentioned examples, and the storage medium storing the program codes constitute the present invention.

When the computer executes the program codes that were read, an operating system (OS) or the like, which is running on the computer, executes a part or all of the actual processing operations based on the instructions of the program codes. The case of implementing the functions of the above mentioned examples by these processing operations as well are also included in the scope of the present invention. Furthermore, it is assumed that the program codes read from the storage medium are written to a function extension card inserted into the computer, or to a memory of a function extension unit connected to the computer. Then based on the instructions of the program codes, the CPU or the like included in the function extension card or function extension unit executes a part or all of the actual processing operations, and the functions of the above mentioned embodiment are implemented by this processing. This case is also included in the scope of the present invention. In the case of applying the present invention to this storage medium, the program codes corresponding to the above described flow charts are stored in the storage medium.

### Other Examples

Those skilled in the art can easily construct a new system by combining various techniques of each example described above, therefore such a system created by various combinations is also included in the scope of the present invention.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-096162, filed on May 7, 2014, which is hereby incorporated by reference herein in its entirety.

What is claimed is:

1. An object information acquiring apparatus, comprising:
a bed configured to have a first opening through which an object is inserted;
a probe configured to include a plurality of first acoustic wave detection elements receiving an acoustic wave propagated from the object and outputting a first reception signal, and a breast support member supporting the plurality of first acoustic wave detection elements so that a direction in which reception sensitivity of at least a part of the plurality of first acoustic wave detection elements is the highest, and a direction in which reception sensitivity of the first acoustic wave detection elements different from the part of the first acoustic wave detection elements is the highest, are different and are both oriented to a predetermined area, the probe being separated from the object, which is inserted through the first opening of the bed, in the normal direction of an opening surface of the first opening;
a plurality of second acoustic wave detection elements which are disposed between the bed and the probe, and of which highest sensitive direction is oriented to the predetermined area, and moreover which receive the acoustic wave and output a second reception signal;
a first position controller configured to change a positional relationship between the object and the predetermined area by changing a positional relationship between the object and the probe; and
a generator configured to generate object information based on the first and second reception signals.

2. The object information acquiring apparatus according to claim 1, wherein
the plurality of first acoustic wave detection elements are disposed along an approximately spherical crown shape.

3. The object information acquiring apparatus according to claim 1, wherein
the breast support member is formed in an approximately spherical crown shape having a second opening.

4. The object information acquiring apparatus according to claim 3, wherein
the predetermined area is formed at or near the center of an approximately spherical shape, which is partially constituted by the approximately spherical crown shape.

5. The object information acquiring apparatus according to claim 4, wherein
the first position controller changes the positional relationship between the object and the probe, so that the center is included in the object which is inserted through the first opening of the bed.

6. The object information acquiring apparatus according to claim 3, wherein
the first position controller implements scanning with the probe, and
the second acoustic wave detection elements are disposed between a scanning surface drawn by an opening surface of the second opening during the scanning and the bed.

7. The object information acquiring apparatus according to claim 1, wherein
the first opening is circular, and
the second acoustic wave detection elements are disposed along the circumference of the first opening.

8. The object information acquiring apparatus according to claim 1, wherein
the bed holds the second acoustic wave detection elements.

9. The object information acquiring apparatus according to claim 7, further comprising a second position controller configured to implement scanning with the second acoustic wave detection elements by changing the positions of the second acoustic wave detection elements along the circumferential direction of the first opening.

10. The object information acquiring apparatus according to claim 1, further comprising an attitude controller configured to orient the directions of the second acoustic wave detection elements, in which the reception sensitivity is the highest, to the predetermined area.

11. The object information acquiring apparatus according to claim 1, further comprising a light irradiator configured to irradiate light to the object in order to propagate the acoustic wave from the object.

12. The object information acquiring apparatus according to claim 1, further comprising an ultrasonic transmitter configured to transmit an ultrasonic wave to the object in order to propagate the acoustic wave from the object.

13. The object information acquiring apparatus according to claim 1, further comprising a display configured to display the generated object information as an image.

14. An object information acquiring apparatus, comprising:
a bed configured to have a first opening through which an object is inserted;
a probe configured to include a plurality of first acoustic wave detection elements receiving an acoustic wave propagated from the object and outputting a first reception signal, and a breast support member supporting the plurality of first acoustic wave detection elements so that orientation axes of the plurality of first acoustic wave detection elements are collected, the probe being separated from the object, which is inserted through the first opening of the bed, in the normal direction of an opening surface of the first opening;
a plurality of second acoustic wave detection elements which are disposed between the bed and the probe, and of which highest sensitive direction is oriented to a predetermined area, and moreover which receive the acoustic wave and output a second reception signal;
a first position controller configured to change a positional relationship between the object and the predetermined area by changing a positional relationship between the object and the probe; and
a generator configured to generate object information based on the first and second reception signals.

15. An breast information acquiring apparatus, comprising:
a bed having an opening through which an breast is inserted, wherein the bed is configured to support a examinee;
a breast support member configured to be located below the opening and connected to the bed so as to form an upper space in which the breast can be inserted and a lower space in which an acquiring unit is located,
wherein the acquiring unit comprises;
a probe unit located below the breast support member and be movably arranged respect to the opening, wherein the probe unit having a moving acoustic wave detection element which detects an acoustic wave propagated from the breast, and having a moving bowl-shaped support member on which the moving acoustic wave detection element is fixed in concave portion such that the moving acoustic wave detection element and the moving bowl-shaped support member are integrally moved respect to the opening;
a fixed acoustic wave detection element fixed on the bed and located at a position between the bed and the probe unit so that a highest sensitive direction of the fixed acoustic wave detection element and a highest sensitive direction of the moving acoustic wave detection element overlap each other;
a position controller configured to change a relative position between the breast support member and the probe unit; and
a generator configured to generate object information based on a detecting signal of the moving acoustic wave detection element and a detecting signal of the fixed acoustic wave detection element.

16. The breast information acquiring apparatus according to claim 15, wherein
the breast support member is annularly connected to the bed.

17. The breast information acquiring apparatus according to claim 15, wherein
the breast support member is arranged to have a front aspect and a back aspect in vertical direction, wherein the front aspect and the back aspect are on acoustic matching liquid propagating the acoustic wave.

18. The breast information acquiring apparatus according to claim 17, wherein
the acoustic matching liquid is water or liquid which consists primarily of water.

19. The breast information acquiring apparatus according to claim 15, wherein
the breast support member is an acoustic coupling member propagating the acoustic wave.

20. The breast information acquiring apparatus according to claim 19, wherein
the acoustic coupling member is polyethylene terephthalate resin.
